(19) United States
(12) Patent Application Publication
(10) Pub. No.: US 2024/0261243 A1
(43) Pub. Date: Aug. 8, 2024

(54) NITRIC OXIDE DONORS, COMPOSITIONS, AND METHODS OF USE

(71) Applicant: KNOW BIO, LLC, Durham, NC (US)

(72) Inventors: John Kelly Simons, Durham, NC (US); Kyle Kimble, Raleigh, NC (US); Nathan Stasko, Durham, NC (US); Rebecca Anthouard McDonald, Durham, NC (US); Madyson Chambers, Durham, NC (US); Adam Cockrell, Durham, NC (US)

(73) Assignee: KNOW BIO, LLC, Durham, NC (US)

(21) Appl. No.: 18/036,120

(22) PCT Filed: Sep. 3, 2021
(86) PCT No.: PCT/US21/49036

§ 371 Date: May 9, 2023

Related U.S. Application Data
(60) Provisional application No. 63/074,660, filed on Sep. 4, 2020.

(51) Int. Cl. A61K 31/198; A01N 33/26; A01N 51/00; A01N 59/00; A01N 59/16; A01P 1/00; A61K 31/655; A61K 33/24; A61K 47/69; A61L 2/18; A61P 31/04; A61P 31/14

(52) U.S. Cl. CPC A61K 31/198; A01N 33/26; A01N 51/00; A01N 59/00; A01N 59/16; A01P 1/00; A61K 31/655; A61K 33/24; A61K 47/6951; A61L 2/18; A61P 31/04; A61P 31/14; A61L 2202/24

(57) ABSTRACT

Disclosed are compositions comprising NO donors formulated for effectively treating disease states, such as microbial infections, including Covid-19 infections. Mucoadhesive agents, chelating agents, and gallium enable highly efficacious formulations for treating infections, particularly when the nature of the pathogenic species is unknown.

### RELATED APPLICATIONS

The presently disclosed subject matter claims priority and is related to U.S. Provisional Patent Application No. 63,074,660 entitled “NITRIC OXIDE DONORS, COMPOSITIONS, AND METHODS OF USE” and filed on Sep. 4, 2020; the entire disclosure of which is incorporated herein by reference.

### FIELD OF THE INVENTION

The present disclosure relates generally to nitric oxide-releasing compounds and to their use as antimicrobial compounds. Antimicrobial compositions comprising these compounds and their methods of use are also disclosed.

### BACKGROUND

Conventional treatments for microbial infections typically involve systemic administration of antimicrobials (antibiotics or antivirals), which can lead to drug resistance and a number of adverse effects from loss of hearing to gastro-intestinal distress. It would be advantageous to have alternative treatments for microbial infections that used an orthogonal mechanism of action to treat microbial infections.

Nitric oxide is known for having such an orthogonal antimicrobial mechanism of action. While the precise mechanisms by which nitric oxide (NO) kills or inhibits the replication of a variety of intracellular pathogens is not completely understood, reactivity towards iron centers involved in cellular metabolism, the imposition of nitrosative stress, and activation of host immunity are likely implicated. Nitric oxide is also understood to target cysteine proteases (Saura et al., Immunity, Volume 10, Issue 1, 1 Jan. 1999, Pages 21-28). NO S-nitrosylates the cysteine residue in the active site of certain viral proteases, inhibiting protease activity and interrupting the viral life cycle. Since cysteine proteases are critical for virulence or replication of many viruses, bacteria, and parasites, NO can be used to treat microbial infections. While long seen as a potentially beneficial therapeutic, the administration of NO gas via inhalation is difficult and time-consuming and antimicrobial levels are close to therapeutic levels, leaving little safety interval.

It would be advantageous to have pharmaceutical compositions comprising NO donors to deliver antimicrobial concentrations of NO to a patient.

### SUMMARY

Nitric oxide-releasing compounds (also referred to as nitric oxide donors or NO donors) and compositions containing such are disclosed. Nitric oxide, an endogenously produced diatomic free radical, is associated with numerous biological processes, including platelet aggregation and adhesion, vasodilation, wound repair, the immune response, the mediation of angiogenesis, and carcinogenesis. Deficiency of NO can lead to some degree of malfunction of NO-relevant physiological systems. Exogenous NO delivery may be an effective strategy for the resolution of biomedical therapies ranging from cardiovascular diseases, to antibacterial and anticancer therapies. NO delivery can also be used to achieve antimicrobial activity.

Pharmaceutical compositions including a nitric oxide releasing compound and an aqueous solution are provided. The nitric oxide releasing compound may be soluble to at least about 25 mg/ml in the aqueous solution at a physiologically compatible pH. The composition may further includes one or more of a chelating agent, a mucoadhesive agent, or a low molecular weight polyethylene glycol. Exemplary, the nitric oxide releasing compounds include diazeniumdiolates and nitrosothiols. The nitric oxide releasing compound may have a NO release half-life at normal physiological temperature and pH of at least 15 minutes. Certain embodiments include gallium.

In certain embodiments, the small molecule nitric oxide precursors are provided in dilute solutions (e.g., for vaporization and inhalation), and in other embodiments, are provided in the form of gels or viscous liquids.

In certain embodiments of the NO donors described herein, secondary amine scaffold molecules are modified (nitrosated) to form NO donors. Other nitric oxide precursors can be present. For example, one or more thiol moieties can be provided as nitrosothiols, —S(NO) moieties, which release nitric oxide and revert to thiols. One or more primary amines can be converted into a nitric oxide precursor having the following formula:

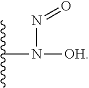 [SMILES: *N(O)N=O]

In various embodiments, the molecular scaffold or precursor includes one or more secondary amine groups, and one or more of these secondary amines can be converted to a NO donating group (e.g. a diazeniumdiolate) as disclosed herein.

In one embodiment, the NO donor is N-(acetyloxy)-3-nitrosothiovaline, also known as S-Nitroso-N-Acetyl-D,L-Penicillamine (SNAP). In another embodiment, the NO donor is 3-(nitrosothio)-N-(1-oxopentyl)-valine, also known as S-Nitroso-N-valeryl-DL-penicillamine. In another embodiment, the NO donor is 3-[2-hydroxy-1-(1-methylethyl)-2-nitrosohydrazinyl]-1-propanamine, also known as NOC-5. In another embodiment, the NO donor is an oxathiazolylium, such as 4-phenyl-1,3,2-oxathiazolidine-5-one, 4-methoxy-4-phenyl-1,3,2-oxathiazolidine-5-one, 4-(p-methoxyphenyl)-1,3,2-Oxathiazolylium-5-olate, or 4-(p-chlorophenyl)-1,3,2-Oxathiazolylium-5-olate. In another embodiment, the NO donor is S-Nitroso-L-glutathione. In another embodiment, the NO donor is (Z)-1-[N-(2-aminoethyl)-N-(2-ammonioethyl)amino]diazen-1-ium-1,2-diolate, also known as Diethylenetriamine NONOate or DETA-NONO, (Z)-1-[N-[3-aminopropyl]-N-[4-(3-aminopropylammonio)butyl]-amino]diazen-1-ium-1,2-diolate, otherwise known as Spermine NONOate, (Z)-1-[N-(3-aminopropyl)-N-(n-propyl)amino]diazen-1-ium-1,2-diolate, also known as PAPA NONOate, or (Z)-1-[N-(3-aminopropyl)-N-(3-ammoniopropyl)amino]diazen-1-ium-1,2-diolate, also known as DPTA NONOate.

In several embodiments, the compound is soluble in water at a concentration of 25 mg/ml. In several embodiments, the compound has a total releasable NO storage in a range of 2-10 μmol of NO per mg of NO donor compound. In several embodiments, the compound has a NO half-life in the range of 0.1-24 hours. In several embodiments, the compound has a total duration of NO release in the range of 1-60 hours. In several embodiments, the total NO release after 4 hours is in the range between 0.1-1.0 μmol of NO per mg of compound.

Methods for treating microbial infections are also disclosed. In various embodiments, an effective amount of the composition comprising one of more of the compounds described herein, alone or in combination with additional components as described herein is administered to a subject in need thereof.

### Methods of Treatment

The NO-releasing compounds described herein are antimicrobial. Representative microbes include viruses, Gram-positive bacteria, Gram-negative bacteria, drug resistant bacteria, molds, yeasts, fungi, and combinations thereof.

Some embodiments pertain to a method of reducing or preventing microbial load on a surface. In several embodiments, the compounds generate nitric oxide and induce oxidative and/or nitrosative damage to microbial DNA and membrane structures, thereby preventing or reducing microbial load. In several embodiments, the plurality of microbes comprises one, two, or more of the following: gram-positive bacteria, gram-negative bacteria, fungi, yeast, and viruses.

In several embodiments, the surface is an organic surface, such as human skin, including epithelial tissue, or a wound surface. In several embodiments, the surface is animal skin. In several embodiments, the application does not induce skin irritation.

In several embodiments, the surface is an inorganic surface. In several embodiments, the inorganic surface is an external or internal surface of a medical device. In several embodiments, the application of the compound generates an anti-microbial coating on the external or internal surface of the medical device. In several embodiments, the medical device comprises an endoscope. The medical device may be a percutaneous continuous glucose monitor, subcutaneous continuous glucose monitor, catheter, prosthetic joint, mechanical heart valve, pacemaker, contact lens, endotracheal tubes, intrauterine devices, or a combination thereof.

### Bacteria

In several embodiments, the microbial load comprises drug-resistant bacteria. In several embodiments, the microbial load comprises microbes associated with the presence of one or more of human immunodeficiency virus, herpes simplex virus, papilloma virus, parainfluenza virus, influenza, hepatitis, Coxsackie Virus, herpes zoster, measles, mumps, rubella, rabies, pneumonia, hemorrhagic viral fevers, H1N1, prions, parasites, fungi, mold, Candida albicans, Aspergillus niger, Escherichia coli, Pseudomonas aeruginosa, and Staphylococcus aureus, Group A streptococci, S. pneumoniae, Mycobacterium tuberculosis, Campylobacter jejuni, Salmonella, Shigella, carbapenem-resistant Enterobacteriaceae Methicillin-resistant Staphylococcus aureus, and Burkholderia cepacia. In several embodiments, the microbial load comprises Methicillin-resistant Staphylococcus aureus. In several embodiments, the microbial load comprises carbapenem-resistant Enterobacteriaceae. In several embodiments, the microbial load comprises Staphylococcus aureus. In several embodiments, the microbial load comprises Pseudomonas aeruginosa. In several embodiments, the microbial load comprises Burkholderia cepacia.

Some embodiments pertain to a method of preventing and/or decreasing microbial contamination. In some embodiments, the method comprises contacting a surface contaminated with a plurality of microbes (or that a surface that could be exposed to microbes) with a NO-releasing scaffold. In some embodiments, a NO-donor of the scaffold generates NO and induces damage to the membrane and/or DNA of the microbes, thereby reducing the number of viable microbes and/or preventing the colonization or infection of an area with microbes. In several embodiments, the surface comprises an organic surface.

In some embodiments of the method, the surface is human skin or animal skin. In some embodiments of the method, the surface is in the mouth, or surrounding tissues (e.g., lips, nasal nares, teeth, gums, etc.). In several embodiments, the surface comprises the oral mucosa. In some embodiments, the surface is any portion of the digestive tract. In some embodiments, the surface is in the lungs or any other part of the respiratory tract.

In some embodiments, the surface is an inorganic surface (of a device, etc.). In several embodiments, the inorganic surface is an external or internal surface of a medical device. In several embodiments, the device is a dental device.

Advantageously, in some embodiments of the method, the application step does not induce skin or tissue irritation. In some embodiments, the plurality of microbes comprises one or more of viruses, Gram-positive bacteria, Gram-negative bacteria, drug resistant bacteria, molds, yeasts, fungi, and combinations thereof. In some embodiments, the surface is an inorganic surface and the application step does not induce oxidation of the inorganic surface.

The compositions and related methods set forth in further detail below describe certain actions taken by a practitioner; however, it should be understood that they can also include the instructions of those actions by another party. Thus, actions such as “administering a NO-donating scaffold” include “instructing the administration of a NO-donating scaffold.”

### BRIEF DESCRIPTION OF THE DRAWINGS

FIG. 1 is a chart showing the results of an HPLC of 2,6 cis-dimethylpiperdine-NONOate.

FIG. 2 is a chart showing the results from a standard TCID₅₀ titration, or plaque assays, of virus harvested at 24-hours and 48-hours post-infection for PAPA NONOate at the specified concentrations.

FIG. 3 is a plot of the viability (raw reads) vs PAPA NONOate concentration (micromolar).

FIG. 4 is a plot of the results from a standard TCID₅₀ titration, or plaque assays, of virus harvested at 24-hours and 48-hours post-infection for SNAP at the specified concentrations (M).

FIG. 5 is a plot of the % Reduction SARS-CoV-2 on a first y-axis and % Cytotoxicity on a second y-axis, demonstrating that SNAP has a SI of approximately 30 towards SARS-CoV-2.

FIG. 6 is a chart showing the MIC of BIOC76 and gallium citrate against P. aeruginosa (PA or PAK), in terms of BIOC76 MIC (mg/ml) vs. gallium citrate (GaCi) concentration (mg/ml).

FIG. 7 is a chart showing the PA time kill for various combinations and concentrations of BIOC76 and/or GaCi, showing CFU/ml of PA versus time (h).

FIG. 8 is a chart showing the PA time kill for a combination of 2 mg/ml BIOC76 and/or GaCi, showing CFU/ml of PA versus time (h).

FIG. 9 is a chart showing the PA time kill for a combination of 4 mg/ml BIOC76 and/or GaCi, showing CFU/ml of PA versus time (h).

FIG. 10 is a chart showing the PA time kill for a combination of 8 mg/ml BIOC76 and/or GaCi, showing CFU/ml of PA versus time (h).

### DETAILED DESCRIPTION

NO-releasing compositions are disclosed. The compositions include a nitric oxide donor selected from NO releasing polymeric materials and NO releasing small molecules. Many NO releasing small molecules are well known in the art. These compounds are substantially different than NO donors. One of the advantages of using small molecules over polymers is that the compounds can be prepared with relatively lower impurity levels than polymeric compounds. Further, relative to polymeric compounds, the NO load may be higher, because the percent composition ratio between NO to the scaffold can be maximized, as described herein. Small molecules can be selected with a low number of reactive amines, reducing the possibility that many different species will result from a nitrosation reaction. As such, the NO-precursor can proceed with little or no partial reaction products, which provides the potential for relatively pure products (i.e., products with a few HPLC peaks).

Knowing the structure of the small molecule allows for more predictable release kinetics than that obtainable with polymers. Additionally, and desirably, the scaffold can be selected such that it also has desirable properties after NO release, i.e., the NO-releasing compound is a prodrug not only for NO, but also for an active metabolite.

Ideally, the compounds have at least one, and, in some embodiments, a single, secondary amine to bear the DAZD or other NO precursor. The remaining scaffold has a relatively low toxicity following NO release. In some embodiments, the NO-releasing compounds release between about 10 and about 20% NO by weight of the NO-releasing compounds. That is, the percent composition of NO within the molecule represents between about 10 and about 20% of the total mass of the NO donor. For example, adding a single diazeniumdiolate group, which carries a mass of about 60 g/mol, would be between 10 and 20% of NO donors with molecular weights of between 300 g/mol and 600 g/mol. In some embodiments, the scaffold has a molecular weight between about 240 g/mol and about 540 g/mol.

In some embodiments, the NO-releasing compounds have a half-life for releasing NO (i.e., an NO half-life) between about 15 minutes and about 48 hours, preferably between about 30 minutes and about 24 hours. DAZD or other NO-releasing moieties typically require intramolecular stabilization to provide this half-life. This stabilization can be provided through pKa (i.e., by including carboxylic acid side chains), intramolecular H-bonding, zwitterionic bonding between the NO-releasing groups and a charged side-chain, protective groups, steric hindrance, and the like.

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Nitric oxide (NO) plays a variety of physiological roles as a signaling molecule and, as disclosed herein, can also play significant roles in treating or ameliorating pathophysiology, for example as a therapeutic agent. NO as a therapeutic has heretofore been underused, based at least in part on limited NO payloads of therapeutic compositions, NO release rates that are more rapid than desired, and the lack of targeted NO delivery. Nitric oxide also has antimicrobial effects. See, e.g., U.S. Patent Application Publication No. 2019/0322770.

The present invention will be better understood with reference to the following definitions.

### Definitions

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

The term “effective amount,” as used herein, refers broadly to that amount of a recited compound that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. For example, an effective amount can refer to the amount of a composition, compound, or agent that improves a condition in a subject by about 5%, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, an improvement in a condition can be a reduction in infection. In some embodiments, an improvement can be reduction of bacterial load (e.g., bioburden) on a surface or in a subject. Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

“Treat” or “treating” or “treatment” refers broadly to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc.

The terms “disrupting” and “eradicating” refer broadly to the ability of the presently disclosed structures to combat biofilms. The biofilms may be partially eradicated or disrupted, meaning that the cells no longer attach to one another or to a surface. The biofilm may be completely eradicated, meaning that the biofilm is no longer an interconnected, cohesive, or continuous network of cells to a substantial degree.

The terms “nitric oxide donor” or “NO donor” refer broadly to species and/or molecules that donate, release and/or directly or indirectly transfer a nitric oxide species, and/or stimulate the endogenous production of nitric oxide in vivo and/or elevate endogenous levels of nitric oxide in vivo such that the biological activity of the nitric oxide species is expressed at the intended site of action.

The terms “nitric oxide releasing” or “nitric oxide donating” refer to species that donate, release and/or directly or indirectly transfer any one (or two or more) of the three redox forms of nitrogen monoxide (NO+, NO−, NO (e.g., ·NO)) and/or methods of donating, releasing and/or directly or indirectly transferring any one (or two or more) of the three redox forms of nitrogen monoxide (NO+, NO−, NO). In some embodiments, the nitric oxide releasing is accomplished such that the biological activity of the nitrogen monoxide species is expressed at the intended site of action.

The term “microbial infection” as used herein refers broadly to bacterial, fungal, viral, yeast infections, as well other microorganisms, and combinations thereof.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects. The subject “in need of” the methods disclosed herein can be a subject that is experiencing a disease state and/or is anticipated to experience a disease state, and the methods and compositions of the invention are used for therapeutic and/or prophylactic treatment.

For the general chemical formulas provided herein, if no substituent is indicated, a person of ordinary skill in the art will appreciate that the substituent is hydrogen. A bond that is not connected to an atom, but is shown, indicates that the position of such substituent is variable. A jagged line, wavy line, two wavy lines drawn through a bond or at the end of a bond indicates that some additional structure is bonded to that position. For a great number of the additional monomers disclosed herein, but not explicitly shown in structures, it is understood by those in the art of polymers, that these monomers can be added to change the physical properties of the resultant polymeric materials even where the elemental analysis would not indicate such a distinction could be expected. Such physical properties include solubility, charge, stability, cross-linking, secondary and tertiary structure, and the like. Moreover, if no stereochemistry is indicated for compounds having one or more chiral centers, all enantiomers and diasteromers are included. Similarly, for a recitation of aliphatic or alkyl groups, all structural isomers thereof also are included. Unless otherwise stated, groups shown as A₁ through Aₙ and referred to herein as an alkyl group, in the general formulas provided herein are independently selected from alkyl or aliphatic groups, particularly alkyl having 20 or fewer carbon atoms, and even more typically lower alkyl having 10 or fewer atoms, such as methyl, ethyl, propyl, isopropyl, and butyl. The alkyl may be optionally substituted (e.g., substituted or not substituted, as disclosed elsewhere herein). The alkyl may be a substituted alkyl group, such as alkyl halide (e.g. —CX₃ where X is a halide, and combinations thereof, either in the chain or bonded thereto), alcohols (e.g. aliphatic or alkyl hydroxyl, particularly lower alkyl hydroxyl) or other similarly substituted moieties such as amino-, amino acid-, aryl-, alkyl aryl-, alkyl ester-, ether-, keto-, nitro-, sulfhydryl-, sulfonyl-, sulfoxide modified-alkyl groups.

The term “amino” and “amine” refer to nitrogen-containing groups such as NR₃, NH₃, NHR₂, and NH₂R, wherein R can be as described elsewhere herein. Thus, “amino” as used herein can refer to a primary amine, a secondary amine, or a tertiary amine. In some embodiments, one R of an amino group can be a diazeniumdiolate (e.g., NONO).

Whenever a group is described as being “optionally substituted” that group may be unsubstituted or substituted with one or more of the indicated substituents. Likewise, when a group is described as being “unsubstituted or substituted” (or “substituted or unsubstituted”) if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, 0-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl), a di-substituted amine(alkyl), a diamino-group, a polyamino, a diether-group, and a polyether-group.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” or “C₁-C₄ alkyl” group refers broadly to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

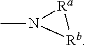

As used herein, the term “alkyl” refers broadly to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers broadly to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are one to five carbon atoms in the alkyl chain, e.g., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers broadly to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] followed by the number of carbon atoms, followed by a “*”. For example,

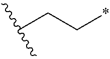 [SMILES: *CC*]

to represent ethylene. The alkylene group may have 1 to 30 carbon atoms (whenever it appears herein, a numerical range such as “1 to 30” refers broadly to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. An alkylene group may be substituted or unsubstituted. For example, a lower alkylene group can be substituted by replacing one or more hydrogens of the lower alkylene group and/or by substituting both hydrogens on the same carbon with a C₃₋₆ monocyclic cycloalkyl group

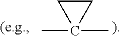

The term “alkenyl” used herein refers broadly to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers broadly to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers broadly to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers broadly to two rings which have two atoms and one bond in common. As used herein, the term “bridged cycloalkyl” refers broadly to compounds wherein the cycloalkyl contains a linkage of one or more atoms connecting non-adjacent atoms. As used herein, the term “spiro” refers broadly to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers broadly to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged, or spiro fashion. A cycloalkenyl group may be unsubstituted or substituted.

As used herein, “aryl” refers broadly to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted. As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers broadly to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer broadly to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “cycloalkyl(alkyl)” refer broadly to an cycloalkyl group connected, as a substituent, via a lower alkylene group. The lower alkylene and cycloalkyl group of a cycloalkyl(alkyl) may be substituted or unsubstituted.

As used herein, “heteroaralkyl” and “heteroaryl(alkyl)” refer broadly to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer broadly to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, the term “hydroxy” refers broadly to a —OH group.

As used herein, “alkoxy” refers broadly to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers broadly to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

As used herein, a “cyano” group refers broadly to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers broadly to a “—C(═S)R” group in which R can be the same as defined with respect to O-carboxy. A thiocarbonyl may be substituted or unsubstituted. An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers broadly to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers broadly to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers broadly to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers broadly to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers broadly to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers broadly to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers broadly to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers broadly to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer broadly to a “—C(═O)OR” group in which R can be the same as defined with respect to O-carboxy. An ester and C-carboxy may be substituted or unsubstituted.

A “nitro” group refers broadly to an “—NO₂” group.

A “sulfenyl” group refers broadly to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers broadly to an “—S(═O)—R” group in which R can be the same as defined with respect to sulfenyl. A sulfinyl may be substituted or unsubstituted.

A “sulfonyl” group refers broadly to an “SO₂R” group in which R can be the same as defined with respect to sulfenyl. A sulfonyl may be substituted or unsubstituted.

As used herein, “haloalkyl” refers broadly to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers broadly to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The terms “amino” and “unsubstituted amino” as used herein refer broadly to a —NH₂ group.

A “mono-substituted amine” group refers broadly to a “—NHR_{A}” group in which R^{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. A mono-substituted amine group can include, for example, a mono-alkylamine group, a mono-C₁-C₆ alkylamine group, a mono-arylamine group, a mono-C₆-C₁₀ arylamine group and the like. Examples of mono-substituted amine groups include, but are not limited to, —NH(methyl), —NH(phenyl) and the like.

A “di-substituted amine” group refers broadly to a “—NRAR_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. A di-substituted amine group can include, for example, a di-alkylamine group, a di-C₁-C₆ alkylamine group, a di-arylamine group, a di-C₆-C₁₀ arylamine group and the like. Examples of di-substituted amine groups include, but are not limited to, —N(methyl)₂, —N(phenyl)(methyl), —N(ethyl)(methyl) and the like.s used herein, “mono-substituted amine(alkyl)” group refers broadly to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, a mono-arylamine(alkyl group), a mono-C₆-C₁₀ arylamine(C₁-C₆ alkyl) group and the like. Examples of mono-substituted amine(alkyl) groups include, but are not limited to, —CH₂NH(methyl), —CH₂NH(phenyl), —CH₂CH₂NH(methyl), —CH₂CH₂NH(phenyl) and the like. As used herein, “di-substituted amine(alkyl)” group refers broadly to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, a di-arylamine(alkyl) group, a di-C₆-C₁₀ arylamine(C₁-C₆ alkyl) group and the like. Examples of di-substituted amine(alkyl) groups include, but are not limited to, —CH₂N(methyl)₂, —CH₂N(phenyl)(methyl), —CH₂N(ethyl)(methyl), —CH₂CH₂N(methyl)₂, —CH₂CH₂N(phenyl)(methyl), —NCH₂CH₂(ethyl)(methyl) and the like.

As used herein, the term “diamino-” denotes a “—N(R_{A})R_{B}—N(R_{C})(R_{D})” group in which R_{A}, R_{C}, and R_{D} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “N” groups and can be (independently of R_{A}, R_{C}, and R_{D}) a substituted or unsubstituted alkylene group. R_{A}, R_{B}, R_{C}, and R_{D} can independently further be substituted or unsubstituted.

As used herein, the term “polyamino” denotes a “—(N(R_{A})R_{B}—)ₙ—N(R_{C})(R_{D})”. For illustration, the term polyamino can comprise —N(R_{A})alkyl-N(R_{A})alkyl-N(R_{A})alkyl-N(R_{A})alkyl-H. In some embodiments, the alkyl of the polyamino is as disclosed elsewhere herein. While this example has only 4 repeat units, the term “polyamino” may consist of 1, 2, 3, 4, 5, 6, 7, 8, 9, or 10 repeat units. R_{A}, R_{C}, and R_{D} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “N” groups and can be (independently of R_{A}, R_{C}, and R_{D}) a substituted or unsubstituted alkylene group. R_{A}, R_{C}, and R_{D} can independently further be substituted or unsubstituted. As noted here, the polyamino comprises amine groups with intervening alkyl groups (where alkyl is as defined elsewhere herein).

As used herein, the term “diether-” denotes an “—OR_{B}O—R_{A}” group in which R_{A} can be a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “O” groups and can be a substituted or unsubstituted alkylene group. R_{A} can independently further be substituted or unsubstituted.

As used herein, the term “polyether” denotes a repeating —(OR_{B}—)ₙOR_{A} group. For illustration, the term polyether can comprise -Oalkyl-Oalkyl-Oalkyl-Oalkyl-OR_{A}. In some embodiments, the alkyl of the polyether is as disclosed elsewhere herein. While this example has only 4 repeat units, the term “polyether” may consist of 1, 2, 3, 4, 5, 6, 7, 8, 9, or 10 repeat units. R_{A} can be a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{B} can be a substituted or unsubstituted alkylene group. R_{A} can independently further be substituted or unsubstituted. As noted here, the polyether comprises ether groups with intervening alkyl groups (where alkyl is as defined elsewhere herein and can be optionally substituted).

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms. As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

When a range of integers is given, the range includes any number falling within the range and the numbers defining ends of the range. For example, when the terms “integer from 1 to 20” is used, the integers included in the range are 1, 2, 3, 4, 5, 6, 7, 8, 9, 10, etc., up to and including 20.

Also as used herein, “and/or” refers broadly to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of 20%, ±10%, +5%, ±1%, ±0.5%, or even 0.1% of the specified amount. The term “consists essentially of” (and grammatical variants), shall be given its ordinary meaning and shall also mean that the composition or method referred to can contain additional components as long as the additional components do not materially alter the composition or method. The term “consists of” (and grammatical variants), shall be given its ordinary meaning and shall also mean that the composition or method referred to is closed to additional components. The term “comprising” (and grammatical variants), shall be given its ordinary meaning and shall also mean that the composition or method referred to is open to contain additional components.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Nitric Oxide Donors

The difficulty in delivering NO as a therapeutic warrants, in several embodiments, the use of assorted synthetic NO donors (e.g., N-diazeniumdiolates, S-nitrosothiols, metal nitrosyls, organic nitrates), in order to control NO delivery. N-diazeniumdiolates (NONOates) may be useful as NO donors because of their good stability and their capacity for proton-triggered NO delivery under physiological conditions. It has a relatively short biological half-life (seconds) and is reactive in nature. In several embodiments disclosed herein, the NO donor comprises any one of the following nitric oxide releasing moieties:

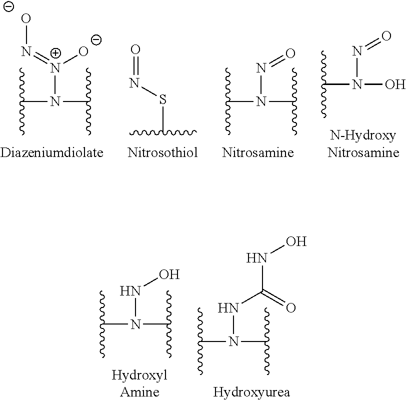

where “ [symbol] ” indicates attachment to other atoms within the structure (e.g., any instance of —H, —CH₂—, —CH—, etc.). In some embodiments, the NO donor is a N-diazeniumdiolate NO donor. In some embodiments, the NO donor is attached to a secondary amine as disclosed elsewhere herein. In other embodiments, such as the nitrosothiol and N-hydroxy nitrosamine moieties, the NO donor is attached to a thiol or primary amine moiety.

The synthesis of scaffolds capable of controlled NO storage and release is important for taking advantage of NO's role in physiology and for developing NO-based therapeutics. In addition to the effects of NO disclosed above, NO is also a potent antibacterial agent that acts on bacteria via nitrosative and/or oxidative stress. NO is a broad-spectrum antibacterial agent and in some embodiments, scaffolds that deliver NO are capable of eradicating both bacteria and biofilms, potentially through the formation of reactive NO byproducts (e.g., peroxynitrite and dinitrogen trioxide) that cause oxidative and nitrosative damage to microbial DNA and/or membrane structures. Advantageously, the wide range of mechanisms by which NO exerts its antibacterial effects reduces the risk that bacteria will develop resistance. Thus, NO-releasing materials may be good targets to battle bacterial infection. The antibacterial efficacy of NO-releasing materials is dependent on both NO payloads and associated release kinetics. In some instances, high NO total is an important parameter to effectively evaluate storage capability of good scaffolds. Additionally, in several embodiments disclosed herein, a high density of secondary amine groups imbues certain donors with a high NO storage capacity. However, NO release that is too fast and high NO storage may result in undesired toxicity to mammalian cells. Therefore, challenges exist in preparing biocompatible NO-releasing materials with high NO storage and low cytotoxicity, and such challenges, among others, are addressed according to several embodiments disclosed herein.

N-diazeniumdiolates NO scaffolds are pH-triggered NO-release donors. Reacting with proton in the physiological condition (e.g., 37° C., pH 7.4), 1 mole of N-diazeniumdiolate regenerates 1 mole of the parent secondary amine compounds and two moles of NO radicals. The real-time detection of NO was performed by using a chemiluminescence-based nitric oxide analyzer (NOA). The total NO storage and dissociation kinetics of water-soluble NO Donors were measured in physiological condition (pH 7.40, 37° C.). The resulting NO-release parameters (e.g., total NO storage, half-life of NO release, maximum flux, and conversion efficiency) were evaluated for suitability as a pharmaceutical product. In general, small molecule derivatives of many compounds can be designed to exhibit large NO storage capabilities with consistent NO-release kinetics (e.g., species which consistently have NO-release half-lives from 0.5 h to 24+h).

Several embodiments disclosed herein have one or more of the following advantages: efficient and unique synthesis routes and resultant chemical composition of small molecules. Controllable amounts of secondary-amines and diverse exterior terminal groups (e.g., hydroxyl, methyl, hydroxymethyl, and primary amine) can be provided. The NO storage and NO-release kinetics of the generated nitric-oxide releasing scaffolds can be tuned for a particular application. This tuning is achieved, in several embodiments, by altering the type and/or number of side chains on the small molecules described herein.

In several embodiments, additional functionalization of the amines in the generated nitric-oxide releasing scaffolds, for example, by compounds with different compositions, further enables the control over NO-release kinetics. In some embodiments, the secondary amine group directly influences the stability of the N-diazeniumdiolate (or other NO carrier group), allowing for control over both NO storage and release kinetics.

As disclosed elsewhere herein, nitric oxide not only plays fundamental roles in several important biological processes, but also exhibits function as an antibacterial or anticancer agent. As disclosed elsewhere herein, various NO donors (e.g., N-diazeniumdiolates, S-nitrosothiols, metal nitrosyls, organic nitrates) can be used for controlled exogenous NO delivery. N-bound diazeniumdiolates are attractive because of their good stability and facile storage, which spontaneously undergo proton-triggered dissociation under physiological condition to regenerate the NO radicals. In several embodiments, progress has been made in preparing and testing biocompatible N-diazeniumdiolate-modified scaffolds, including those derived from biopolymers and saccharide derived polymers (e.g., chitosan, hyaluronic acid, CMC, etc.).

As disclosed elsewhere herein, some embodiments disclosed herein pertain to the use of small molecule scaffolds to deliver NO to achieve microbicidal activity. In some embodiments, the small molecule itself has antimicrobial or other desired physiological properties. In some embodiments, the scaffold is water soluble.

In several embodiments, the scaffolds are functionalized with one or more instances of each of R₁, R₂, R₃, R₄, R₅, and R₆. In several embodiments, each instance of R₁, R₂, R₃, R₄, R₅, and R₆ are independently selected from the group consisting of —OH, —NH₂, —OCH₃, —C(O)OH, —CH₂OH, —CH₂OCH₃, —CH₂OCH₂CH₂OH, —OCH₂C(O)OH, —C H₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)ₐNH)ₑ—H, —C(O)—NH—((CH₂)ₐNH)ₑ—(CH₂)_{f}H, —C(O)—X¹—((CH₂)_{g}X²)ₕ—(CH₂)ᵢH, —C(O)—X¹—((CH₂)_{g}X²)ₕ((CH₂)ⱼX³)ₖ—(CH₂)ᵢH, —O—((CH₂)ₐO)_{b}—H, —O—((CH₂)ₐ O)_{b}—(CH₂)_{c}H, —O—(C₁₋₅alkyl), —NH—((CH₂)ₐNH)ₑ—H, —NH—((CH₂)_{d}NH)ₑ—(CH₂)_{f}H, —X¹—((CH₂)_{g}X²)ₕ—(CH₂)ᵢH, —X¹—((CH₂)_{g}X²)ᵢ((CH₂)ⱼX³)ₖ—(CH₂)ᵢH, wherein each instance of a, b, c, d, e, f, g, h, i, j, k, and 1 is independently selected from an integer of 0, 1, 2, 3, 4, 5, 6, 7, 8, 9, or 10, wherein, each instance of X¹, X², and X³ is independently selected from —O—, —S—, —NH—, C(O)NH—; and wherein at least one instance of X¹, X², and X³ is represented by one of the following NO donating groups

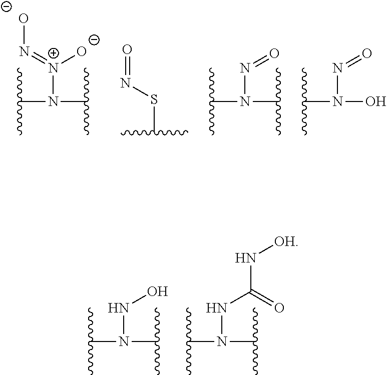

II. Scaffolds with Mono-Diazeniumdiolates

In some embodiments, the compounds described herein are designed to release nitric oxide following administration, and that portion of the compounds that is not the diazeniumdiolate (DAZD) moiety comprise a secondary amine group, which amine group is

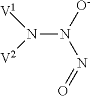

wherein V¹ and V² are variables 1 and 2. These variables are, independently, selected to provide one of two properties, namely, steric hindrance to stabilize the diazeniumdiolate (DAZD) functional group, and to stabilize the DAZD functional group via hydrogen bonding and/or ionic bonding (i.e., an ionic bond between the O⁻ on the DAZD moiety and an ammonium salt, or hydrogen bonding between the uncharged oxygen or one of the nitrogens in the DAZD moiety and the hydrogen in a moiety that includes an OH, NH, SH, SO₂H, SO₃H, or CO₂H functional group).

The DAZD group is generally understood to provides 2 moles of nitric oxide per single DAZD. Ideally, the scaffold (i.e., that portion of the molecule that remains after evolution of nitric oxide) is non-toxic or minimally toxic. In order to maximize the amount of nitric oxide per dosage of compound administered, it is desired that the scaffold be relatively small, with a target of 10-20% NO by weight of the compound. This can be accomplished, for example, where the scaffold more typically between about 240 and about 540 g/mol. In other embodiments, a scaffold may have molecular weight of between about 100 and about 750 g/mol.

In one embodiment, the compounds have the following formula:

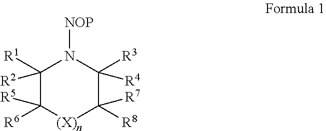

wherein NOP stands for “nitric oxide precursor, examples of which include the following:

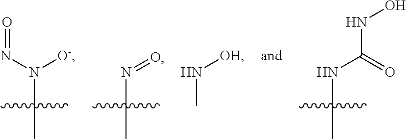

R¹-R⁸ are, independently, H, C₁₋₆ alkyl, C₂₋₆ alkenyl, C₁₋₆ alkynyl, C₃₋₈ cycloalkyl, C₃0.8 heterocyclyl, arylalkyl, alkylaryl, aryl, heteroaryl, alkylheteroaryl, heteroarylalkyl, —C(V)VH, —C(V)V⁻M⁺, C₁₋₆ alkylamine, C₁₋₆ hydroxyalkyl, C₁₋₆ thioalkyl, C₁₋₆ sulfonate, and C₁₋₆ alkyl-V-C₁₋₆ alkyl,
wherein one or both of R² and R⁶ and/or R⁴ and R⁸ can form a double bond between the two carbons to which they are linked, and
wherein one or both of R¹, R², R⁵ and R⁶, and R³, R⁴, R⁷ and R⁸ together form an aryl or heteroaryl ring, which ring(s) can optionally be substituted with one, two, or three substituents Z,
Z is, independently, C₁₋₆ alkyl, C₃₋₈ cycloalkyl, C₂₋₆ alkenyl, C₂₋₆ alkynyl, heterocyclyl, aryl, heteroaryl, halo (e.g., F, Cl, Br, or I), —OR′, —NR′R″, —CF₃, —CN, —NO₂, —C₂R′, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, and —NR′SO₂R″, where R′ and R″ are individually hydrogen, C₁₋₆ alkyl, C₃₋₈ cycloalkyl, heterocyclyl, aryl, heteroaryl, or arylalkyl (such as benzyl),
R′ and R″ are, independently, H, C₁₋₆ alkyl, C₁₋₆ haloalkyl, C₁₋₆ alkoxy, C₃₋₈ cycloalkyl, C₂₋₆ alkenyl, C₂₋₆ alkynyl, heterocyclyl, aryl, heteroaryl, alkylaryl or arylalkyl, wherein these groups can optionally be substituted with one or more substituents Z as defined above,
wherein V is, independently, O, S, or NR⁹, where R⁹ is H, C₁₋₆ alkyl, C₂₋₆ alkenyl, C₁₋₆ alkynyl, C₃₋₈ cycloalkyl, C₃₋₈ heterocyclyl, arylalkyl, alkylaryl, aryl, heteroaryl, alkylheteroaryl, or heteroarylalkyl, and where M is an alkali metal, such as lithium, sodium, or potassium, though other metals can be used, so long as the charges balance (i.e., one divalent metal ion for two —C(V)V⁻ ions, with the proviso that at least one of R¹-R⁴ is other than H, and
wherein X is, independently, C(V), C(R⁵)₂, N(R⁹), O, or S(O)ₘ,
n is 0, 1, 2, or 3, and
m is 0, 1 or 2.

In one aspect of this embodiment, the compounds have the following formula:

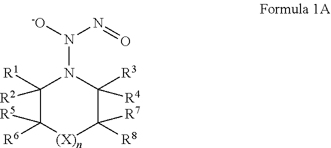

where R¹-R⁹, X, Z, V, M, R′, R″, n and m are as defined above.

In one embodiment, all of R² and R⁴-R⁸ are H.

In one embodiment, R¹ and R³ are C₁₋₆ alkyl.

In one embodiment, one of R¹ and R³ is C₁₋₆ alkyl, and the other is a moiety selected from the group consisting of —C(V)VH, —C(V)V⁻M⁺, C₁₋₆ alkylamine, C₁₋₆ hydroxyalkyl, C₁₋₆ thioalkyl, and C₁₋₆ sulfonate.

In one embodiment, R¹ and R³ are C₁₋₆ alkyl, such as methyl, R² and R⁴-R⁸ are H, X is NR⁹ and n is 1. In one aspect of this embodiment, the compound has the following structure:

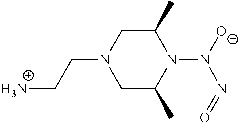

In another embodiment, R¹ is C₁₋₆ alkyl, such as methyl, R³ is a carboxylic acid or carboxylate salt (i.e., —C(V)VH or —C(V)V⁻M⁺, where each V is O), R² and R⁴-R⁸ are H, X is NR⁹ n is 1, and R⁹ is a C₁₋₆ alkylamine, C₁₋₆ hydroxyalkyl, C₁₋₆ thioalkyl, or C₁₋₆ sulfonate, such as a propylamine side chain. In this embodiment, where the compounds are alpha-disubstituted cyclic amines, the compounds have doubly flanked DAZD groups, one group providing steric hindrance, and the other providing the potential for intramolecular H-bonding and/or ionic bonding. Further, the other ring nitrogen includes a side chain, such as an alkylamine side chain, that also provides the potential for intramolecular H-bonding and/or ionic bonding. One representative compound is shown below:

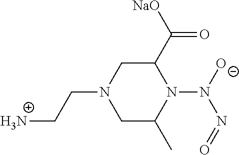

While not wishing to be bound to a particular theory, it is believed that providing a carboxylic acid group at this position reduces the mutagenic potential of resulting nitrosamines.

In one embodiment, the precursor compound is an azacyclic ring which includes two ring nitrogens, both of which are secondary amines, and both of which are flanked with substituents that will stabilize a DAZD group, whether through steric hindrance, hydrogen bonding, ionic bonding, or combinations thereof. In this embodiment, each compound will have two stabilized DAZD moieties.

In another aspect of this embodiment, R¹ is C₁₋₆ alkyl, such as methyl, or C₁₋₆ alkylamine, such as propylamine, R³ is C₁₋₆ alkylamine, such as propylamine, R² and R⁴-R⁸ are H, X is CH₂, and n is 1. Representative compounds are shown below:

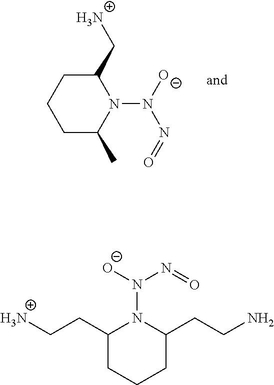

In this aspect, the ring is relatively simplified, by only including one ring nitrogen, and the compounds include one or more intramolecular H-bonding or ionic bonding flanking side groups at the R¹ and/or R³ positions.

In another aspect, the compounds include a seven-member ring with two ring nitrogens (i.e., 1,4-diazepines), with doubly-flanked DAZD groups, which can provide steric hindrance, hydrogen bonding and/or ionic bonding to the DAZD group. The second ring nitrogen can also include a side chain that provides hydrogen bonding and/or ionic bonding to the DAZD group. For example, R¹ and R³ can be C₁₋₆ alkyl, such as methyl, n is 2, one of X is CH₂ and the other is NR⁹, where R⁹ is a C₁₋₆ alkylamine, such as a propylamine/propylammonium salt, for example, a N,N-dimethyl propylammonium salt.

A representative 1,4-diazepine is shown below:

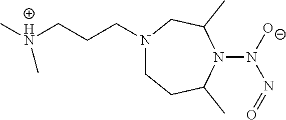

Other representative precursor compounds (which can be converted to DAZD analogs) include the following:

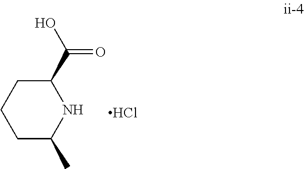

(2s,6s)-6-methylpipecolic acid, which, when the ring nitrogen is converted to a DAZD group, is expected to have a half life less than 30 minutes.

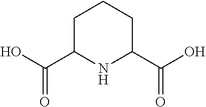 [SMILES: O=C(O)C1CCCC(C(=O)O)N1]

Piperidine-2,6-dicarboxylic acid, which, when the ring nitrogen is converted to a DAZD group, is expected to have a half life less than 10 minutes.

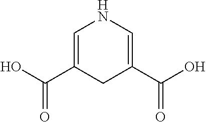 [SMILES: O=C(O)C1=CNC=C(C(=O)O)C1]

3,5-Pyridinedicarboxylic acid, which falls within Formula 1 when both of R² and R⁶ and R⁴ and R⁸ form a double bond between the two carbons to which they are attached.

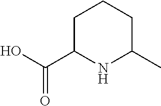

6-methylpiperidine-2-carboxylic acid

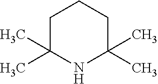

2,2,6,6-Tetramethylpiperidine, which, when the ring nitrogen is converted to a DAZD group, is believed to have a half-life>1 hr.

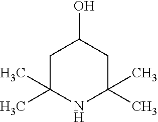

2,2,6,6-Tetramethyl-4-piperidinol, which, when the ring nitrogen is converted to a DAZD group, is believed to have a half-life>1 hr.

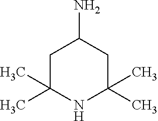

4-Amino-2,2,6,6-tetramethyl-piperidine, which, when the ring nitrogen is converted to a DAZD group, is believed to have a half-life>1 hr.

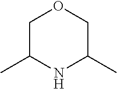

3,5-Dimethylmorpholine, which, when the ring nitrogen is converted to a DAZD group, is believed to have a half-life of around 1 hr.

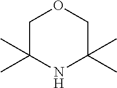

3,3,5,5-tetramethylmorpholine, which, when the ring nitrogen is converted to a DAZD group, is believed to have a half-life of around 1.5 hr.

In another embodiment, sugars are used as a scaffold. The sugars are modified to include a secondary amine side chain, which side chain can also be functionalized with an C₁₋₆ alkyl-carboxylic acid moiety, or salt thereof, a C₁₋₆ hydroxyalkyl moiety, or a C₁₋₆ thioalkyl moiety. In one aspect of this embodiment, the secondary amine side chain is provided at a position alpha to the ring oxygen. These moieties can not only provide steric hindrance to stabilize the resulting DAZD moieties, but also provide hydrogen bonding and/or ionic binding to the DAZD moieties.

In some aspects of this embodiment, the sugars are monosaccharides, and in others, are disaccharides.

Monosaccharides are classified according to three different characteristics: the placement of its carbonyl group, the number of carbon atoms it contains, and its chiral handedness. If the carbonyl group is an aldehyde, the monosaccharide is an aldose; if the carbonyl group is a ketone, the monosaccharide is a ketose. Monosaccharides with three carbon atoms are called trioses, those with four are called tetroses, five are called pentoses, six are hexoses, and so on. These two systems of classification are often combined. For example, glucose is an aldohexose (a six-carbon aldehyde), ribose is an aldopentose (a five-carbon aldehyde), and fructose is a ketohexose (a six-carbon ketone).

Each carbon atom bearing a hydroxyl group (—OH), with the exception of the first and last carbons, are asymmetric, making them stereo centers with two possible configurations each (R or S). Because of this asymmetry, a number of isomers may exist for any given monosaccharide formula. Using Le Bel-van′t Hoff rule, the aldohexose D-glucose, for example, has the formula (CH₂O)₆, of which four of its six carbons atoms are stereogenic, making D-glucose one of 24=16 possible stereoisomers.

In the case of glyceraldehydes, an aldotriose, there is one pair of possible stereoisomers, which are enantiomers and epimers. 1,3-dihydroxyacetone, the ketose corresponding to the aldose glyceraldehydes, is a symmetric molecule with no stereo centers. The assignment of D or L is made according to the orientation of the asymmetric carbon furthest from the carbonyl group: in a standard Fischer projection if the hydroxyl group is on the right the molecule is a D sugar, otherwise it is an L sugar. The “D-” and “L-” prefixes should not be confused with “d-” or “l-”, which indicate the direction that the sugar rotates plane polarized light. This usage of “d-” and “1-” is no longer followed in carbohydrate chemistry.

Two joined monosaccharides are called a disaccharide. In some embodiments, the sugars are, other than the presence of the secondary amine side chain, naturally-occurring sugars. Exemplary sugars that can be modified to include the secondary amine side chain include the following:

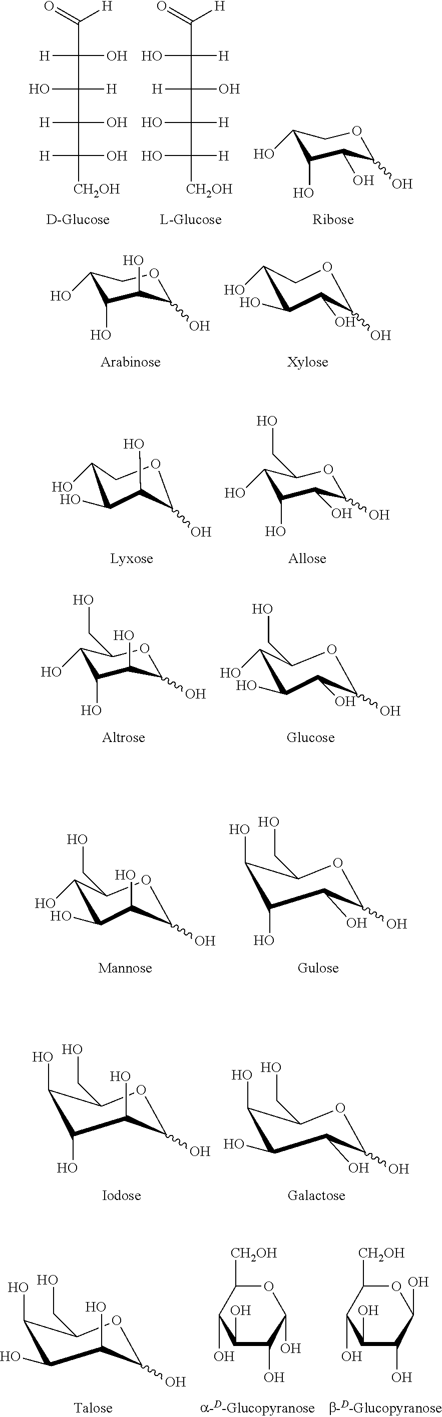

Amino sugars, such as galactosamine, glucosamine, sialic acid and N-acetylglucosamine, can also be used.

Representative modified sugars, which can serve as precursors to DAZD-containing compounds, include the following:

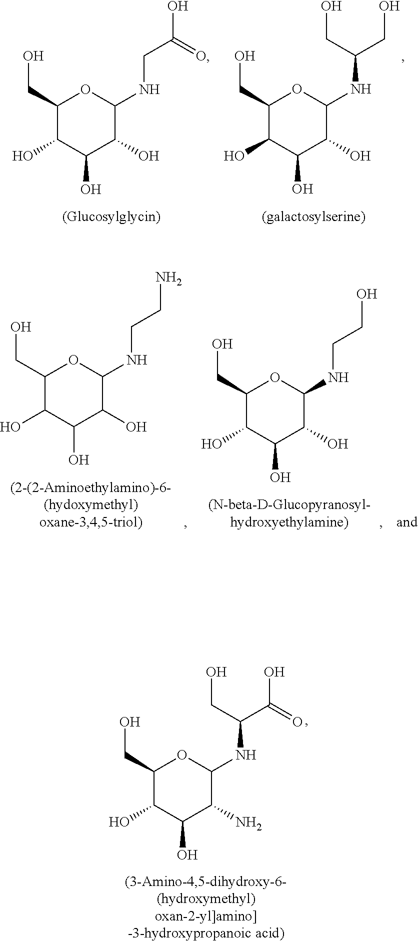

### Azetidines and an Example of a Combination Scaffold/Chelator

Azetidines are another class of compounds that can be used as scaffolds. One such scaffold is nicotiamine, a naturally occurring phytosiderophore. Nicotianamine has a single primary amine, a single secondary amine, and a single tertiary amine (azetidine) linked by propyl groups with a carboxylic acid separated from each amine with a single carbon. The compound has the following formula:

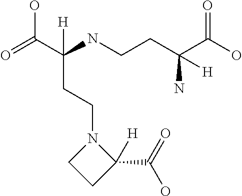 [SMILES: N[C@@H](CCN[C@@H](CCN1CC[C@H]1C(=O)O)C(=O)O)C(=O)O]

Nicotianamine can be reacted with two molecules of nitric oxide (NO) in the presence of a base, such as calcium hydroxide, to yield a nicotianamine analog with two molecules of nitric oxide attached to the secondary amine.

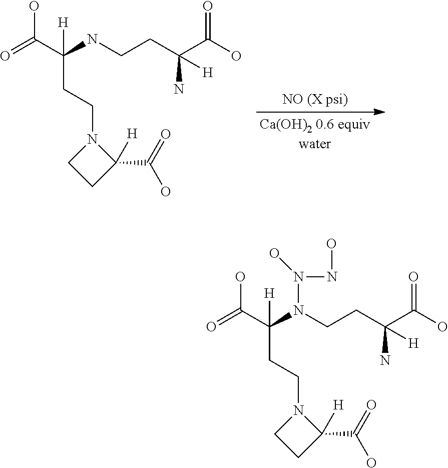 [SMILES: N[C@@H](CCN[C@@H](CCN1CC[C@H]1C(=O)O)C(=O)O)C(=O)O ; N[C@@H](CCN([C@@H](CCN1CC[C@H]1C(=O)O)C(=O)O)N(O)NO)C(=O)O]

Nicotianamine is a novel angiotensin-converting enzyme 2 (ACE2) inhibitor present in soybean and many other plants where it is a siderophore involved in metal transport (Takahashi et al., “Nicotianamine is a novel angiotensin-converting enzyme 2 inhibitor in soybean,” Biomed Res. 2015; 36(3):219-24. doi: 10.2220/biomedres.36.219. PMID: 26106051). An IC₅₀ value of 84 nM was the first demonstration of an ACE2 inhibitor from foodstuffs. ACE2 has been shown to be a co-receptor for viral entry for SARS-CoV-2 with increasing evidence that it has a protracted role in the pathogenesis of COVID-19 (Zhou et al., “A pneumonia outbreak associated with a new coronavirus of probable bat origin,” Nature, 2020 Mar; 579(7798):270-273. ACE2 has a broad expression pattern in the human body with strong expression noted in the gastrointestinal system, heart, and kidney with more recent data identifying expression of ACE2 in type II alveolar cells in the lungs. Nicotianamine (NA) is a natural chelator (a phytosiderophore) of Fe, Zn, and other metals in higher plants and NA-chelated Fe is highly bioavailable in vitro. Already present in our diet, it is being studied for enriching food with Fe and Zn by upregulating NAS expression (Beasley, et al., “Nicotianamine-chelated iron positively affects iron status, intestinal morphology and microbial populations in vivo (Gallus gallus),” Sci Rep 10, 2297 (2020).

Potential features:
Anti-viral: SARS-CoV-2 entry inhibition through ACE2 inhibition
Dual inhibition by blocking ACE2 receptor and nitrosating spike proteins.
Anti-microbial synergy: NO disturbs Fe metabolism and Fe-scavenging compounds have been discovered to be additive or synergistic in combination with NO Donors.
Gallium Synergy—the observed synergy between NO and Ga is expected to be enhanced with Fe scavenging as it shifts equilibrium towards Ga insertion and inactivated Fe-centers.

### Establishing NO Release Kinetics

The following table represents a series of NO donors (mono-diazeniumdiolate compounds) that inform the selection of appropriate small molecule donors as will be described herein.

TABLE 1
Representative Diazeniumdiolate NO Donors

| | | NO Release t ½ (min) | | |
|---|---|---|---|---|
| | | 37° C. at | 22° C. at | |
| Entry | Compound | pH 7.4 | pH 7.4 | Reference |
| 1 | 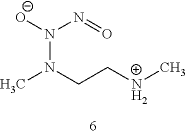 | ND | 36.1 | 1 |
| 2 | 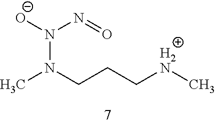 | ND | 10.1 | 1 |
| 3 | 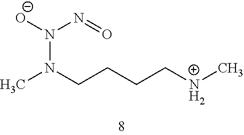 | ND | 1.3 | 1 |
| 4 | 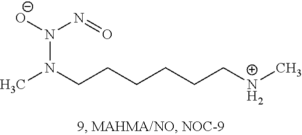 | 1 | 2.7 | 1, 6 |
| 5 | 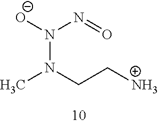 | ND | 40 | 1 |
| 6 | 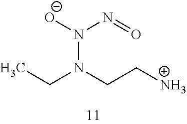 | ND | 333 | 1 |
| 7 | 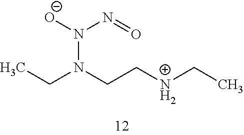 | ND | 327 | 1 |
| 8 | 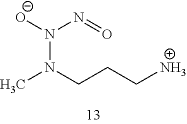 | ND | 13.7 | 1 |
| 9 | 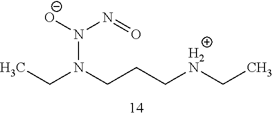 | ND | 71.8 | 1 |
| 10 | 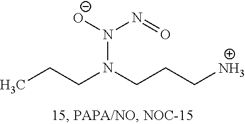 | 8 | 76.6 | 1 |
| 11 | 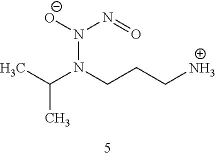 | ND | 93.0 | 1 |
| 12 | 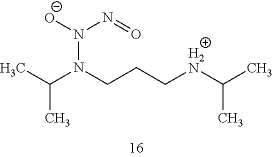 | ND | 88.5 | 1 |
| 13 | 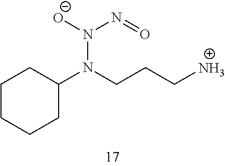 [SMILES: [NH3+]CCCN(C1CCCCC1)N([O-])N=O] | ND | 115 | 1 |
| 14 | 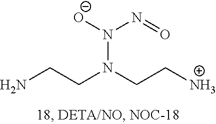 [SMILES: NCCN(CC[NH3+])N([O-])N=O] | 1200 | 3400 | 1 |
| 15 | 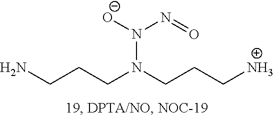 [SMILES: NCCCN(CCC[NH3+])N([O-])N=O] | 180 | 284 | 1, 2 |
| 16 | 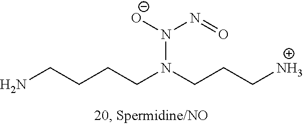 [SMILES: NCCCCN(CCC[NH3+])N([O-])N=O] | ND | 165 | 1 |
| 17 | 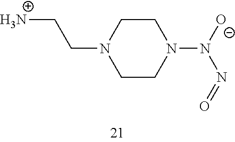 [SMILES: [NH3+]CCN1CCN(N([O-])N=O)CC1] | ND | 5.0 | 1 |
| 18 | 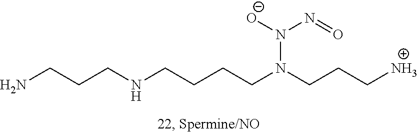 [SMILES: NCCCNCCCCN(CCC[NH3+])N([O-])N=O] | 39 | 230 | 1 |
| 19 | 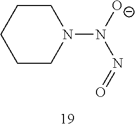 | 0.6, 0.3 | ND | 2, 3 |
| 20 | 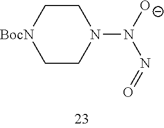 [SMILES: CC(C)(C)OC(=O)N1CCN(N([O-])N=O)CC1] | 2.9 | ND | 3 |
| 21 | 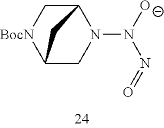 [SMILES: CC(C)(C)OC(=O)N1C[C@H]2C[C@@H]1CN2N([O-])N=O] | 0.3 | ND | 3 |
| 22 | 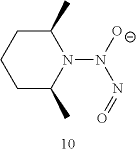 | 37.9 | ND | 3 |
| 23 | 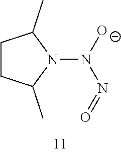 | 2.2 | ND | Error! Bookmark not defined. |
| 24 | 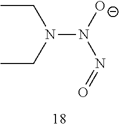 | 2.9, 1.9 | 16 | 2, 3, 4 |
| 25 | 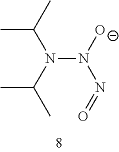 | 3.3 | ND | 3 |
| 26 | 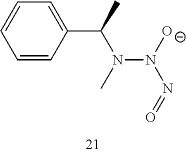 | 3.7 | ND | 3 |
| 27 | 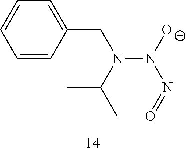 | 7.3 | ND | 3, 4 |
| 28 | 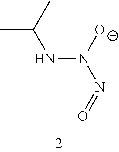 | 3.6 | ND | 5 |
| 29 | 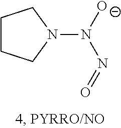 | 0.05, 0.06 | ND | 2, 4 |
| 30 | 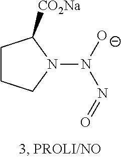 | 0.03 | ND | 6 |
| 31 | 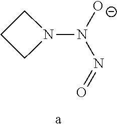 | 1.4 | ND | 4 |
| 32 | 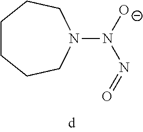 [SMILES: O=NN([O-])N1CCCCCC1] | 0.06 | ND | 4 |
| 33 | 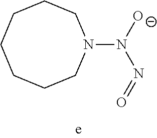 [SMILES: O=NN([O-])N1CCCCCCC1] | 0.07 | ND | 4 |
| 34 | 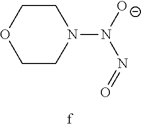 | 2.5 | ND | 4 |
| 35 | 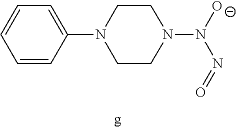 [SMILES: O=NN([O-])N1CCN(c2ccccc2)CC1] | 4.4 | ND | 4 |
| 36 | 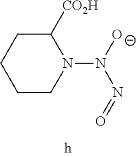 [SMILES: O=NN([O-])N1CCCCC1C(=O)O] | 0.1 | ND | 4 |
| 37 | 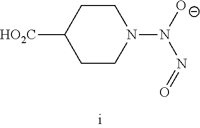 [SMILES: O=NN([O-])N1CCC(C(=O)O)CC1] | 0.4 | ND | 4 |
| 38 | 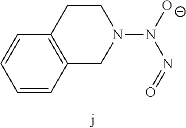 [SMILES: O=NN([O-])N1CCc2ccccc2C1] | 0.5 | ND | 4 |
| 39 | 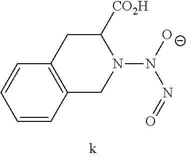 [SMILES: O=NN([O-])N1Cc2ccccc2CC1C(=O)O] | 0.1 | ND | 4 |
| 40 | 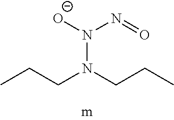 | 1.6 | ND | 4 |
| 41 | 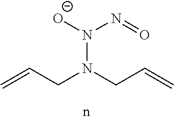 | 4.1 | ND | 4 |
| 42 | 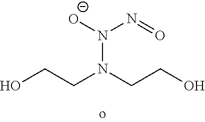 [SMILES: O=NN([O-])N(CCO)CCO] | 7.6 | ND | 4 |
| 43 | 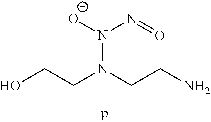 [SMILES: NCCN(CCO)N([O-])N=O] | 125 | ND | 4 |
| 44 | 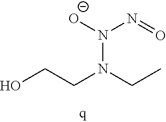 | 3.4 | ND | 4 |
| 45 | 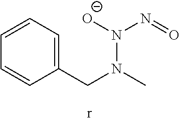 | 0.9 | ND | 4 |
| 46 | 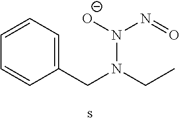 | 5.0 | ND | 4 |
| 47 | 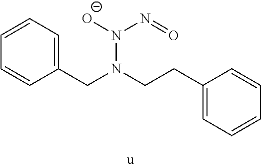 [SMILES: O=NN([O-])N(CCc1ccccc1)Cc1ccccc1] | 9.0 | ND | 4 |
| 48 | 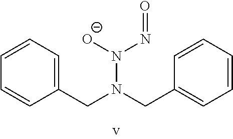 [SMILES: O=NN([O-])N(Cc1ccccc1)Cc1ccccc1] | 11.3 | ND | 4 |
| 49 | 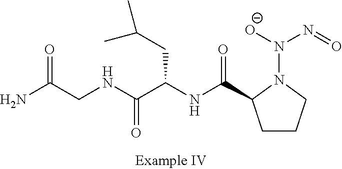 [SMILES: CC(C)C[C@H](NC(=O)[C@@H]1CCCN1N([O-])N=O)C(=O)NCC(N)=O] | 7 | ND | 2 |
| 50 | 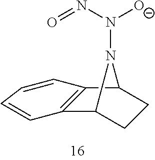 [SMILES: O=NN([O-])N1C2CCC1c1ccccc12] | 17.8 | ND | 3 |

The references are as follows:
1. J. Org. Chem. 1993, 58, 1472-1476
2. Methods in Enzymology 1996, 268, 281-293
3. Org. Lett. 2019, 21, 4210-4214
4. Eur. J. Org. Chem. 2007, 616-624
5. J. Med. Chem. 1991, 34, 3242-3247
6. ACS Chem. Biol. 2011, 6, 1147-1155
7. WO1996015797A1
8. Bioorg. Med. Chem. Lett. 2013, 23, 2769-2774
9. Org. Lett. 2007, 9, 4551-4554

The rate of nitric oxide release from diazeniumdiolates can be controlled using a variety of structural features. Table 1 provides data regarding the release rate of nitric oxide. The data are reported as the diazeniumdiolate half-life in aqueous mediate at pH 7.4 at either 37° C. or 22° C. From the data in Table 1, a number of factors impacting release rate can be established.

The presence of a second amine to form a zwitterion provides an opportunity for an intramolecular hydrogen bond to form, which is proposed to stabilize the diazeniumdiolate anion (see structure A in Scheme 1). The impact of the H-bonding stabilization decreases with increasing chain length n (compare entries 1-4 in Table 1). A chain length of two carbons provides an diazeniumdiolate half-life of 36.1 minutes at 22° C. Increasing the chain length to three decreases the half-life to 10.1 minutes. Further increasing the chain length to 4-5 decreases the half-life to 1.3-2.7 min.

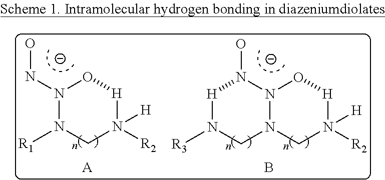

The presence of a third amine can provide the opportunity for a second intramolecular hydrogen bond (see structure B in Scheme 1) and further increased stabilization. Compare Table 1 entry 5 to entry 14 and entry 2 to entries 15, 16, and 18. The addition of a third amine can increase release half-life by up to 85-fold.

In addition to amine substituents, adjacent alcohols can also provide enhanced diazeniumdiolate stability, although to a lesser extent. For example, comparison of Table 1 entry 24 to entries 44 and 42 shows that incorporation of a single alcohol to the diazeniumdiolate of diethylamine results in a modest increase in the diazeniumdiolate half-life from 1.9 minutes (entry 24) to 3.4 min (entry 44). Addition of a second alcohol further lengthens the half-life to 7.6 minutes (entry 42). Combinations of amines and alcohols can be used to further tune the NO release rate. For example, exchanging one of the alcohols in entry 42 with an amine (entry 43) increases the release half-life to 125 minutes.

Stability can also be enhanced through the incorporation of steric bulk around the diazeniumdiolate (ie, R₁ in structure A in Scheme 1). For example, exchange of a methyl with an ethyl substituent can decrease the NO release rate by 8-fold (compare Table 1 entry 5 to entry 6). Entries 8, 10, and 11 show an increase in release half-life from 13.7 minutes to 76.6 minutes to 93.0 minutes in going from methyl to n-propyl to i-propyl substituents. Increasing the bulk around cyclic amines also increases the corresponding diazeniumdiolate stability. This can be seen with both piperidine (entry 19 vs 22) and pyrrolidine (entry 29 vs 23). Addition of flanking methyl groups can increase the release half-life by over 60-fold.

The ring size for cyclic amines has an impact on the NO release rate for diazeniumdiolates. The relative stability for each ring size follows the trend 4>6>5=7=8. This can be observed by comparing Table 1 entries 31, 19, 29, 32, and 33; entry 22 to entry 23; and entry 20 to entry 21.

The pKa of the amine (N³-nitrogen) bearing the diazeniumdiolate has been proposed to play a role in the rate of NO release. The mechanism for NO release from a diazeniumdiolate is believed to involve an initial protonation of the N³-nitrogen (Scheme 2). Decomposition of the protonated intermediate provides 2 equivalents of NO and the corresponding amine.

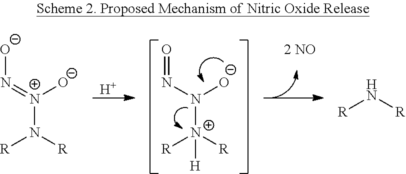

Impeding the initial protonation by lowering the pKa of the N³-nitrogen is believed to slow the rate of NO release. For example, the pKa for N-methyl-cis-2,6-dimethylpiperidine (9.35) is lower than that of the unsubstituted N-methylpiperidine (10.08), suggesting a steric effect on the protonation of N-substituted piperidines. As has already been shown, the rate of NO release from the diazeniumdiolate of cis-2,6-dimethylpiperidine (Table 1, entry 22) is over 60-fold slower than from the corresponding unsubstituted piperidine (entry 19). The difference in pKa could help account for the change in release rate. In addition to steric bulk, the pKa of piperdine can be lowered through the incorporation of a second heteroatom in the amine ring. For example, the pKa of morpholine and piperazine are lower than piperdine. As can be seen in Table 2, the lower pKa values of morpholine and N-phenylpiperazine are accompanied by a reduced NO release rate from the corresponding diazeniumdiolates.

TABLE 2
Amine pKa and NO Release Rate

| Amine | 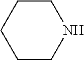 | 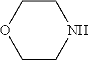 | 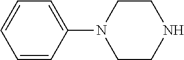 [SMILES: c1ccc(N2CCNCC2)cc1] |
|---|---|---|---|
| Amine | 11.1 | 8.5 | 9.5 |
| pKa | | | |
| Diazen- | 0.3 | 2.5 | 4.4 |
| iumdio- | | | |
| late NO | | | |
| Release | | | |
| t_{1/2} | | | |
| (min) | | | |

Incorporation of a carboxylate group alpha to the diazeniumdiolate may increase the rate of NO release. This can be seen by comparing Table 1 entry 30 to entry 29, entry 36 to entry 19, and entry 39 to entry 38. In these examples, the carboxylate-substituted analogs were approximately two- to five-fold less stable than the unsubstutited analogs. Interestingly, conversion of the carboxylate to an amide has the ability to increase the stability of the diazeniumdiolate. For example, the proline amide analog in Table 1 entry 49 has a diazeniumdiolate half-life over 200-fold longer than the proline carboxylate analog (entry 30) and 140-fold longer than the unsubstituted pyrrolidine analog (entry 29).

The 7-azabenzobicyclo[2.2.1]heptane structure also has the ability to improve diazeniumdiolate stability. For example, the 7-azabenzobicyclo[2.2.1]heptane diazeniumdiolate in Table 1 entry 50 is 8-fold more stable than the monocyclic dimethyl-substituted pyrrolidine analog in entry 23.

In illustrative embodiments, the NO donor is a polymeric NO donor. For instance, a non-derivatized polymer chain having one or more hydroxyl, amino, or carboxyl functional groups, can be functionalized and/or derivatized via those functional groups to add, for example, one or more of R₁, R₂, R₃, R₄, R₅, and R₆. Thus, the disclosed methods are applicable to any biocompatible polymer having one or more of these functional groups pendant from the polymer chain. In several embodiments, the polymer is a biopolymer. In several embodiments, the polymer is a biodegradable polymer. In several embodiments, the polymer is a polysaccharide. In several embodiments the polysaccharide comprises a polymer derived from chitosan, hyaluronic acid, carboxymethylcellulose, hydroxyethyl cellulose, methyl cellulose, cellulose, alginate, cyclodextrin, aminoglycosides, or other polysaccharide. In several embodiments the polysaccharide comprises one or more of the following structures:

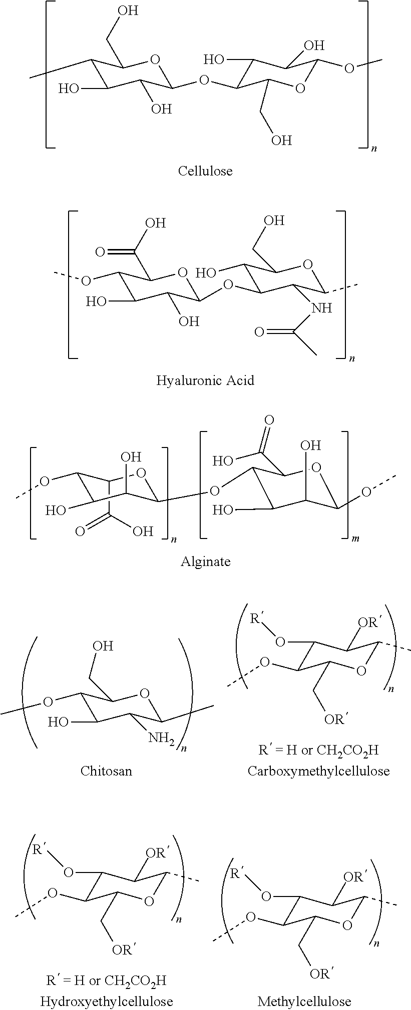

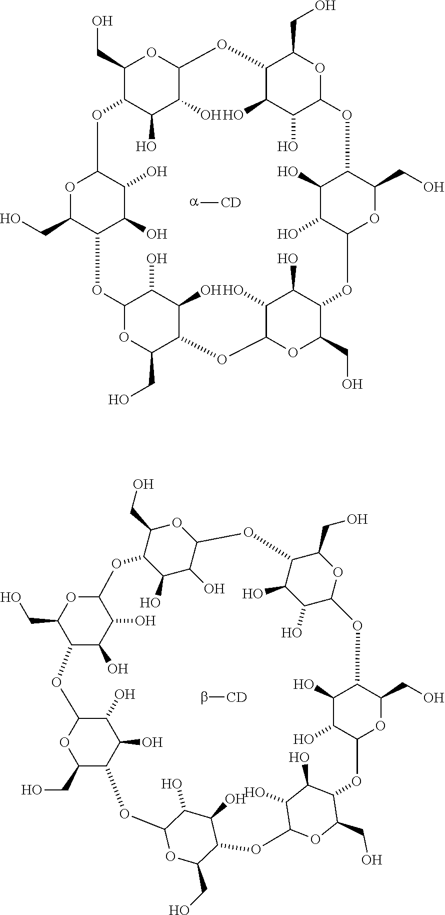

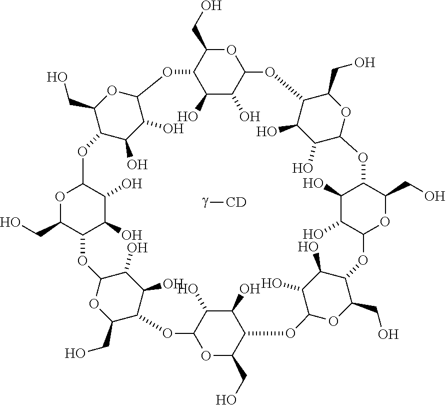

One substituted cyclodextrin is BIOC76, which is a P3-cyclodextrin with seven —CH₂—OH groups that are converted to —CH₂NHCH₂CH₂OH groups, and the secondary amine groups are then reacted with gaseous NO to form diazeniumdiolate groups. The chemistry is summarized below. For the conversion of the hydroxy groups on the cyclodextrin, the hydroxy groups can be converted to any suitable leaving group, such as a tosylate group, then the tosylate can be displaced using conventional nucleophilic displacement chemistry to form the intermediate (CD-EOH₇). Conditions for reacting secondary amines with nitric oxide to form diazeniumdiolates are well known to those of skill in the art.

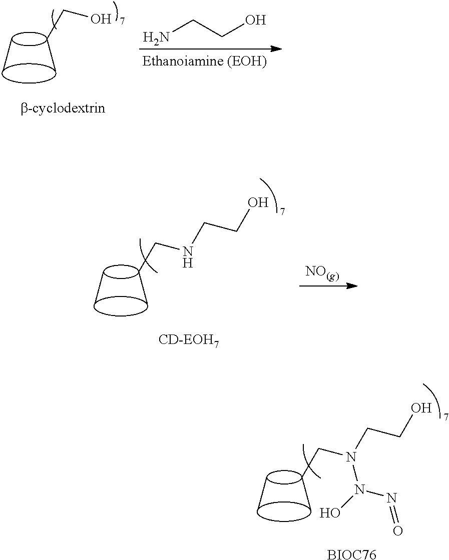

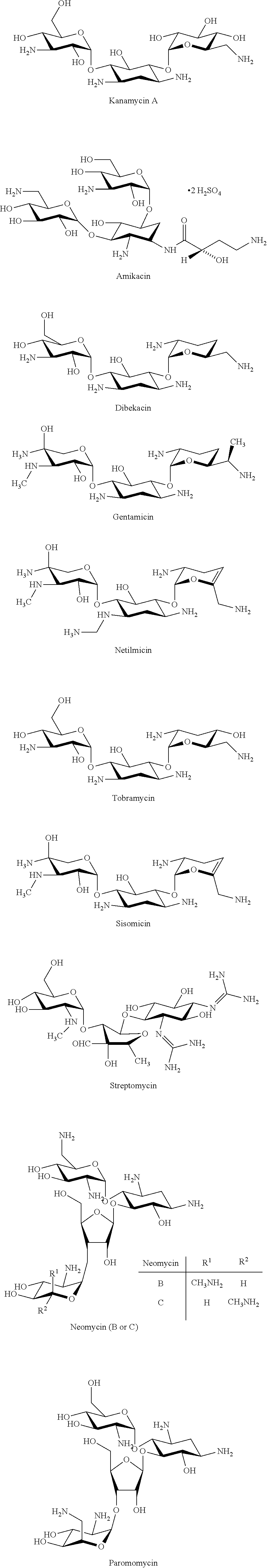

where any one or more of the hydroxyl, amino, or carboxyl functional groups shown above, can be functionalized or derivatized via those functional groups to add, for example, one or more of R₁, R₂, R₃, R₄, R₅, and R₆. In some embodiments, any one of the amino groups of an aminoglycoside could be functionalized with a linking unit (as disclosed in PCT/IB2018/052144, published as WO/2018/178902, which is hereby incorporated by reference in its entirety) to prepare a macromolecular structure.

In several embodiments, the NO-releasing compounds are stable at a variety of temperatures 20° C. (e.g., 400 C, 45° C., 550 C, 60° C., 80° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year or greater)).

In some embodiments, the NO-releasing small molecules have NO storage capacities (in μmol NO/mg compound) of greater than or equal to about: 0.25, 0.4, 0.5, 1.0, 1.5, 2.0, 3.0, or ranges including and/or spanning the aforementioned values. In some embodiments, within 2 h of being added to a PBS buffer solution as described in the Examples, the NO-releasing compounds release greater than or equal to about: 25%, 50%, 75%, 85%, 90%, 95%, 100%, or ranges including and/or spanning the aforementioned values, their total wt % of bound NO. In several embodiments, NO release in use for reducing or eliminating a biofilm occurs in similar amounts, e.g., about 20-25%, about 30-50%, about 60-75%, at least 80%, at least 85%, at least 90%, at least 95%, ranges including and/or spanning the aforementioned values, of the total wt % of bound NO.

In some embodiments, the NO release may occur over a period of about 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In several embodiments, the NO release half-life is equal to or at least about: 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In some embodiments, nitrosamine is not present during NO release. As used herein the phrase “nitrosamine is not present” refers broadly to levels of nitrosamine which are not detectable as determined by a UV-vis spectrum (or by other accepted methods in the art).

In some embodiments, the disclosed NO-releasing compounds have a degradation rate per hour in an amylase enzyme exposure assay of less than or equal to about: 0.2%, 0.5%, 1.0%, 1.5%, 2.5%, 5.0%, 10%, or ranges including and/or spanning the aforementioned values.

In some embodiments, the disclosed functionalized NO-releasing compounds have antimicrobial activity. In some embodiments, the disclosed functionalized NO-releasing compounds provide greater than or equal to 90% bacterial reduction in a bacterial viability assay performed under static conditions over 2 hours against one or more of P. aeruginosa, S. aureus P. gingivalis, A. actinomycetemcomitans, A. viscosus, and/or S. mutans at a concentration of equal to or less than about: 8 mg/ml, 6 mg/ml, 4 mg/ml, 2 mg/ml, 1 mg/ml, 0.5 mg/ml, or ranges including and/or spanning the aforementioned values. In some embodiments, the disclosed functionalized NO-releasing compounds provide greater than or equal to 99% bacterial reduction and/or a 2 to 3 log reduction in a bacterial viability assay performed under static conditions over 2 hours against a gram positive bacteria at a polymer concentration of equal to or less than about: 8 mg/ml, 6 mg/ml, 4 mg/ml, 2 mg/ml, 1 mg/ml, 0.5 mg/ml, or ranges including and/or spanning the aforementioned values. In some embodiments, the disclosed functionalized NO-releasing compounds provide greater than or equal to 99% bacterial reduction and/or a 2 to 3 log reduction in a bacterial viability assay performed under static conditions over 2 hours against a gram negative bacteria at a concentration of equal to or less than about: 8 mg/ml, 6 mg/ml, 4 mg/ml, 2 mg/ml, 1 mg/ml, 0.5 mg/ml, or ranges including and/or spanning the aforementioned values. In several embodiments, bacterial reduction is greater than 95%, greater than 98%, or greater than 99%.

### III. Compound Synthesis

Several embodiments disclosed herein provide the synthesis and characterization of N-diazeniumdiolate NO donor-modified scaffolds and their use in antimicrobial applications. In some embodiments, the scaffolds comprise one or more saccharide units and/or are mono-, di-, or tri-saccharide.

The scaffolds can efficiently eradicate or reduce the viability of microbes (e.g., prokaryotic cells, bacteria, protozoa, fungi, algae, amoebas, slime molds, etc., including drug-resistant microbes) with low toxicity native tissue and patient cells (e.g., eukaryotic cells, mammalian cells, human cells, etc.).

The synthesis ofnicotianamine is disclosed, for example, in U.S. Publication 20020122857 by Asai, et al., which is hereby incorporated by reference in its entirety.

The synthesis involves the precipitation fractionation of an aqueous extract of soybeans by adding an organic solvent such as ethanol, or the molecular weight fractionation of the extract by ultrafiltration or size exclusion chromatography. Either approach is combined with activated carbon filtration, cation- or anion-exchange resin treatment or other adsorbent (e.g., polyamide or octadecylsilica) treatment to provide a nicotianamine product of desired purity.

U.S. Publication No. 20030087410 discloses a nicotianamine synthetase, and a gene encoding the synthase. The gene can be used to prepare a vector containing this gene, which can be used to transfect cells. Nicotianamine can then be produced by those cells, and plants can be transformed to include the gene encoding the nicotianamine synthase.

The synthase has the following sequence:

Met Asp Ala Gln Asn Lys Glu Val Ala Ala Leu
Ile Glu Lys Ile Ala Gly Ile Gln Ala Ala Ile
Ala Glu Leu Pro Ser Leu Ser Pro Ser Pro Glu
Val Asp Arg Leu Phe Thr Asp Leu Val Thr Ala
Cys Val Pro Pro Ser Pro Val Asp Val Thr Lys
Leu Ser Pro Glu His Gln Arg Met Arg Glu Ala
Leu Ile Arg Leu Cys Ser Ala Ala Glu Gly Lys
Leu Glu Ala His Tyr Ala Asp Leu Leu Ala Thr
Phe Asp Asn Pro Leu Asp His Leu Gly Leu Phe
Pro Tyr Tyr Ser Asn Tyr Val Asn Leu Ser Arg
Leu Glu Tyr Glu Leu Leu Ala Arg His Val Pro
Gly Ile Ala Pro Ala Arg Val Ala Phe Val Gly
Ser Gly Pro Leu Pro Phe Ser Ser Leu Val Leu
Ala Ala His His Leu Pro Glu Thr Gln Phe Asp
Asn Tyr Asp Leu Cys Gly Ala Ala Asn Glu Arg
Ala Arg Lys Leu Phe Gly Ala Thr Ala Asp Gly
Val Gly Ala Arg Met Ser Phe His Thr Ala Asp
Val Ala Asp Leu Thr Gln Glu Leu Gly Ala Tyr
Asp Val Val Phe Leu Ala Ala Leu Val Gly Met
Ala Ala Glu Glu Lys Ala Lys Val Ile Ala His
Leu Gly Ala His Met Val Glu Gly Ala Ser Leu
Val Val Arg Ser Ala Arg Pro Arg Gly Phe Leu
Tyr Pro Ile Val Asp Pro Glu Asp Ile Arg Arg
Gly Gly Phe Glu Val Leu Ala Val His His Pro
Glu Gly Glu Val Ile Asn Ser Val Ile Val Ala
Arg Lys Ala Val Glu Ala Gln Leu Ser Gly Pro
Gln Asn Gly Asp Ala His Ala Arg Gly Ala Val
Pro Leu Val Ser Pro Pro Cys Asn Phe Ser Thr
Lys Met Glu Ala Ser Ala Leu Glu Lys Ser Glu
Glu Leu Thr Ala Lys Glu Leu Ala Phe.

Biochemical production of this compound is disclosed, for example, in Yasuaki WADA, Kobayashi et al., “Metabolic Engineering of Saccharomyces cerevisiae Producing Nicotianamine: Potential for Industrial Biosynthesis of a Novel Antihypertensive Substrate,” Bioscience, Biotechnology, and Biochemistry, 70:6, 1408-1415 (2006).

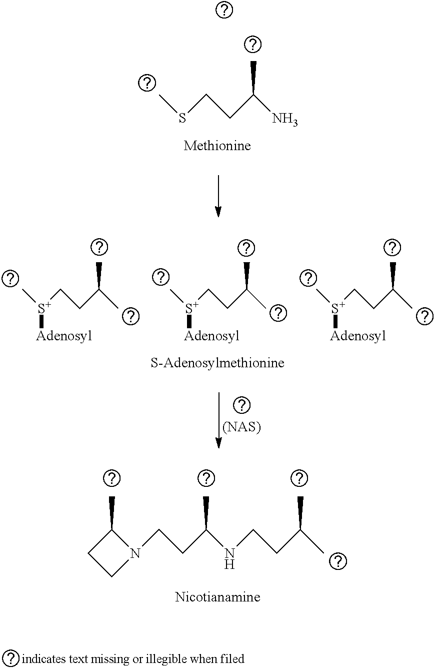

The loading of small molecules with diazeniumdiolate using calcium hydroxide has been demonstrated, and the resulting compounds typically have relatively high purity (see, for example, Zhang et al., Org. Lett. 2019, 21, 11, 4210-4214).

### IV. Compositions

In some embodiments, the compositions can take the form of, for example, tablets or capsules prepared by a conventional technique with pharmaceutically acceptable excipients, such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The pharmaceutically acceptable excipient may be a binding agent, filler, lubricant, disintegrant, wetting agent, suspending agent, solubilizer, or a mixture thereof.

The tablets can be coated by methods known in the art. For example, a therapeutic agent can be formulated in combination with hydrochlorothiazide, and as a pH stabilized core having an enteric or delayed release coating which protects the therapeutic agent until it reaches the target organ.

The compositions may be in the form of a liquid, dry powder, gel, or aerosol. The compositions may be provided in the form of loaded into a delivery device, such as an inhaler.

In some embodiments, the composition includes a concentration of less than or equal to about: 1 mg/ml, 10 mg/ml, 20 mg/ml, 50 mg/ml, 100 mg/ml, 250 mg/ml of the compounds described herein, or ranges including and/or spanning the aforementioned values.

### Formulations for Pulmonary Administration

In some embodiments, the compounds are administered to the pulmonary tract (i.e., via pulmonary administration). In one specific embodiment, pulmonary administration comprises inhalation of the compounds, typically in the form of particles or droplets, such as by nasal, oral inhalation, or both. The particles or droplets can be administered in two or more separate administrations (doses).

In one aspect of this embodiment, particles may be formulated as an aerosol (i.e.: liquid droplets of a stable dispersion or suspension of particles which include one or more of the compounds described herein in a gaseous medium). Particles delivered by aerosol may be deposited in the airways by gravitational sedimentation, inertial impaction, and/or diffusion. Any suitable device for generating the aerosol may be used, including but not limited to pressured meter inhalers (pMDI), nebulizers, dry powder inhalers (DPI), and soft-mist inhalers.

In one specific embodiment, the methods comprise inhalation of particles including one or more of the compounds described herein aerosolized via nebulization. Nebulizers generally use compressed air or ultrasonic power to create inhalable aerosol droplets of the particles or suspensions thereof. In this embodiment, the nebulizing results in pulmonary delivery to the subject of aerosol droplets of the particles or suspension thereof.

In another embodiment, the methods comprise inhalation of particles aerosolized via a pMDI, wherein the particles or suspensions thereof are suspended in a suitable propellant system (including but not limited to hydrofluoroalkanes (HFAs) containing at least one liquefied gas in a pressurized container sealed with a metering valve. Actuation of the valve results in delivery of a metered dose of an aerosol spray of the particles or suspensions thereof.

Biodegradable particles can be used for the controlled-release and delivery of the compounds described herein. Aerosols for the delivery of therapeutic agents to the respiratory tract have been developed. Adjei, A. and Garren, J. Pharm Res. 7, 565-569 (1990); and Zanen, P. and Lamm, J.-W. J. Int. J. Pharm. 114, 111-115 (1995).

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. Gonda, I. “Aerosols for delivery of therapeutic and diagnostic agents to the respiratory tract,” in Critical Reviews in Therapeutic Drug Carrier Systems 6:273-313, 1990. The deep lung, or alveoli, are the primary target of inhaled therapeutic aerosols for systemic drug delivery.

Accordingly, it can be important to deliver antiviral particles to the deep lung (i.e., the alveolar regions of the lung). Relatively large particles tend to get trapped in the oropharyngeal cavity, which can lead to excessive loss of the inhaled drug. Relatively smaller particles can be delivered to the deep lung, but can be phagocytosed. One way to deliver relatively large particles (sized to avoid phagocytosis), which are light enough to avoid excessive entrapment in the oropharyngeal cavity, is to use porous particles.

In one embodiment, the particles for delivering the compounds described herein to the alveolar regions of the lung are porous, “aerodynamically-light” particles, as described in U.S. Pat. No. 6,977,087. Aerodynamically light particles can be made of a biodegradable material, and typically have a tap density less than 0.4 g/cm³ and a mass mean diameter between 5 m and m. The particles may be formed of biodegradable materials such as biodegradable polymers. For example, the particles may be formed of a functionalized polyester graft copolymer consisting of a linear alpha-hydroxy-acid polyester backbone having at least one amino acid group incorporated herein and at least one poly(amino acid) side chain extending from an amino acid group in the polyester backbone. In one embodiment, aerodynamically light particles having a large mean diameter, for example greater than 5 m, can be used for enhanced delivery of one or more of the compounds described herein to the alveolar region of the lung.

### Nanoparticulate Compositions

The compounds described herein can also be administered in the form of nanoparticulate compositions.

In one embodiment, the controlled release nanoparticulate formulations comprise a nanoparticulate active agent to be administered and a rate-controlling polymer which functions to prolong the release of the agent following administration. In this embodiment, the compositions can release the active agent, following administration, for a time period ranging from about 2 to about 24 hours or up to 30 days or longer. Representative controlled release formulations including a nanoparticulate form of the active agent are described, for example, in U.S. Pat. No. 8,293,277.

Nanoparticulate compositions comprise particles of the active agents described herein, having a non-crosslinked surface stabilizer adsorbed onto, or associated with, their surface.

The average particle size of the nanoparticulates is typically less than about 800 nm, more typically less than about 600 nm, still more typically less than about 400 nm, less than about 300 nm, less than about 250 nm, less than about 100 nm, or less than about 50 nm. In one aspect of this embodiment, at least 50% of the particles of active agent have an average particle size of less than about 800, 600, 400, 300, 250, 100, or 50 nm, respectively, when measured by light scattering techniques.

A variety of surface stabilizers are typically used with nanoparticulate compositions to prevent the particles from clumping or aggregating. Representative surface stabilizers are selected from the group consisting of gelatin, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, poloxamines, poloxamine 908, dialkylesters of sodium sulfosuccinic acid, sodium lauryl sulfate, an alkyl aryl polyether sulfonate, a mixture of sucrose stearate and sucrose distearate, p-isononylphenoxypoly-(glycidol), SA90HCO, decanoyl-N-methylglucamide, n-decyl-D-glucopyranoside, n-decyl-D-maltopyranoside, n-dodecyl-D-glucopyranoside, n-dodecyl-D-maltoside, heptanoyl-N-methylglucamide, n-heptyl-D-glucopyranoside, n-heptyl-D-thioglucoside, n-hexyl-D-glucopyranoside, nonanoyl-N-methylglucamide, n-nonyl-D-glucopyranoside, octanoyl-N-methylglucamide, n-octyl-D-glucopyranoside, and octyl-D-thioglucopyranoside. Lysozymes can also be used as surface stabilizers for nanoparticulate compositions. Certain nanoparticles such as poly(lactic-co-glycolic acid) (PLGA)-nanoparticles are known to target the liver when given by intravenous (IV) or subcutaneously (SQ).

In one embodiment, the nanoparticles or other drug delivery vehicles are targeted to the liver. One such type of liver-targeted drug delivery vehicle is described in Park, et al., Mol Imaging. Feb 2011; 10(1): 69-77, and uses Glypican-3 (GPC3) as a molecular target. Park taught using this target for hepatocellular carcinoma (HCC), a primary liver cancer frequently caused by chronic persistent hepatitis.

In one aspect of this embodiment, this drug delivery vehicle is also used to target therapeutics to the liver to treat viral infections. Further, since the compounds described herein have anti-cancer uses, this type of system can target the compounds to the liver and treat liver cancers. GPC3 is a heparan sulfate proteoglycan that is not expressed in normal adult tissues, but significantly over-expressed in up to 80% of human HCC's. GPC3 can be targeted, for example, using antibody-mediated targeting and binding (See Hsu, et al., Cancer Res. 1997; 57:5179-84).

Another type of drug delivery system for targeting the liver is described in U.S. Pat. No. 7,304,045. The '045 patent discloses a dual-particle tumor or cancer targeting system that includes a first ligand-mediated targeting nanoparticle conjugated with galactosamine, with the ligand being on a target cell. The first nanoparticle includes poly(y-glutamic acid)/poly(lactide) block copolymers and n antiviral compound, which in this case is a compound described herein, and in the '045 patent, was gancyclovir. A second nanoparticle includes poly(γ-glutamic acid)/poly(lactide) block copolymers, an endothelial cell-specific promoter, and a (herpes-simplex-virus)—(thymidine kinase) gene constructed plasmid, and provides enhanced permeability and retention-mediated targeting. The first and said second nanoparticles are mixed in a solution configured for delivering to the liver. When the disorder to be treated is a liver tumor or cancer, the delivery can be directly to, or adjacent to, the liver tumor or cancer.

Representative rate controlling polymers into which the nanoparticles can be formulated include chitosan, polyethylene oxide (PEO), polyvinyl acetate phthalate, gum arabic, agar, guar gum, cereal gums, dextran, casein, gelatin, pectin, carrageenan, waxes, shellac, hydrogenated vegetable oils, polyvinylpyrrolidone, hydroxypropyl cellulose (HPC), hydroxyethyl cellulose (HiEC), hydroxypropyl methylcelluose (HPMC), sodium carboxymethylcellulose (CMC), poly(ethylene) oxide, alkyl cellulose, ethyl cellulose, methyl cellulose, carboxymethyl cellulose, hydrophilic cellulose derivatives, polyethylene glycol, polyvinylpyrrolidone, cellulose acetate, cellulose acetate butyrate, cellulose acetate phthalate, cellulose acetate trimellitate, polyvinyl acetate phthalate, hydroxypropylmethyl cellulose phthalate, hydroxypropylmethyl cellulose acetate succinate, polyvinyl acetaldiethylamino acetate, poly(alkylmethacrylate), poly(vinyl acetate), polymers derived from acrylic or methacrylic acid and their respective esters, and copolymers derived from acrylic or methacrylic acid and their respective esters.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” and U.S. Pat. No. 6,432,381 for “Methods for targeting drug delivery to the upper and/or lower gastrointestinal tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

### Controlled Release Formulations

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

### Mucoadhesive Agents

Mucoadhesion is presently defined as the adhesion between two materials, at least one of which is a mucosal surface. Compounds, such as NO donors, are often delivered locally because their half-life is often below the time required for systemic distribution. The mucoadhesive agents described herein enable formulations suitable for mucoadhesive drug delivery systems (buccal, nasal, ocular, gastro, vaginal, and rectal). Mucoadhesive-containing topical and local systems have been shown to exhibit enhanced bioavailability. For example, it typically provides enhanced absorption (compared to a non-mucoadhesive formulation) and taking advantage of mucous tissues having high surface area and high blood flow.

In some embodiments, the mucoadhesive agent is a mucoadhesive polymer. In some embodiments, the mucoadhesive agent has numerous hydrophilic groups, such as hydroxyl, carboxyl, amide, and sulfate. These groups enable attachment to mucus or the cell membrane through physical and chemical interactions such as hydrogen bonding, hydrophobic, electrostatic, or conformational interactions. Hydrophilicity augments through drawing water for greater hydration and physically swell if in a gelatinous state. Aspects considered in selecting an appropriate mucoadhesive agent include the following:
1. Toxicity of the mucoadhesive agent and any potential degradation products, NO donor, and nitrosative environment, considering absorbability and mucus surface irritation,
2. Formulation compatibility with the NO donor,
3. Physiochemical mucoadhesive properties with mucus and epithelial cell surfaces (adhesion, kinetics, specificity, impact to pH and osmolality, stability, etc.), and
4. Commercial considerations (regulatory status, cost, availability, etc.).

In one embodiment, the mucoadhesive agent adheres to the mucosal surface through nonspecific, noncovalent interactions which are primarily electrostatic in nature. In another embodiment, the mucoadhesive agent adheres to the mucosal surface through hydrophilic functional groups that hydrogen bond with similar groups on biological substrates. In another embodiment, the mucoadhesive agent adheres to the mucosal surface through specific receptor sites on the cell or mucus surface. For example, lectins and thiolated polymers adhere to mucosal surfaces through specific receptor sites on the cell or mucus surfaces. As used herein, lectins are proteins or glycoprotein complexes of nonimmune origin that are able to bind sugars selectively in a noncovalent manner. It is proposed that lectins attach to carbohydrates on the mucus or epithelial cell surface. Thiolated polymers, or thiomers, have pendant thiols providing hydrophilicity, for example to polyacrylates or cellulosic polymeric backbones. The thiol group may form stable covalent bonds with mucus glycoproteins resulting in increased residency and improved bioavailability.

Many such mucoadhesive agents are known in the art. Useful mucoadhesive polymers include but are not limited to carbopols, N-isopropylacrylamide, polyvinyl alcohol/polyvinyl pyrrolidone, dextran, hydroxyethylmethacrylate/methacrylic acid, polyvinyl alcohol, polyacrylamide, polyethylene glycol/poly lactic acid, carboxymethyl chitosan and collagen. The mucoadhesive agent may include a polycarbophil and other acrylate/methacrylate polymers, anionic polymers based on methacrylic acid esters, which form pH selectably dissolvable hydrogels that dissolve (enabling physiological conditions to interact with and further initiate the release of NO) within physiochemically specified pH ranges, generally between about pH 5.5 to about pH 7.5. Such formulations dissolving in the pH range from about 5.5 to about 6.0 is useful for targeting the duodenum. Dissolution at higher pH generally targets lower sections of the intestine. For example, a pH of dissolution of between about 6.5 to about 7.0 may be useful for targeting the colon.

In some embodiments, the mucoadhesive agent comprises a water-soluble polymer. In particular, while a water-soluble polymer may or may not form a hydrogel to some extent when hydrated, it is capable of forming a flowable aqueous solution. Mucoadhesive agents of this type include but are not limited to polyols and polycarbohydrates, hydroxylated celluloses (hydroxypropylmethyl cellulose and hydroxymethyl cellulose).

In certain embodiments, the mucoadhesive agent enhances resonance time of the nitric oxide donor at the targeted site, for example, the respiratory tract.

In other certain embodiments, the mucoadhesive agent may also possess adhesion specificity to a biofilm comprising a pathogenic species. For example, some alginate oligomers are known to interact with pseudomonas Aeruginosa biofilms.

### Chelating Agent

In illustrative embodiments, compositions disclosed herein may include one or more chelating agents. According to one aspect, a chelating agent is included to scavenge trace metals, so as to quench their potentially deleterious effects on the NO donor compound. For example, copper may adversely modulate the release kinetics of nitrosothiols. As such, a copper chelator may be provided within the formulation to enhance the stability of the pharmaceutical product prior to use. Exemplary chelating agents are known in the art and examples include Ethylenediaminetetraacetic acid (EDTA) or diethylenetriaminepentaacetic acid (DTPA), yet other compounds described by Baldari S, Di Rocco G, Toietta G. Current Biomedical Use of Copper Chelation Therapy. Int J Mol Sci. 2020; 21(3):1069. Published 2020 Feb 6.

Siderophores have been used in medicine for iron and aluminum overload therapy and antibiotics for improved targeting. For example, an iron-chelating microbial siderophore can be conjugated to an antibiotic or antimicrobial agent to enhance uptake and antibacterial potency. Several embodiments disclosed herein pertain to a pharmaceutical formulation comprising at least one nitric oxide releasing moiety, optionally in combination with at least one siderophore. In some embodiments, the at least one siderophore is functionalized by a NO-donating group, and the composition further includes at least one pharmaceutically acceptable excipient.

### Composition Properties

In several embodiments, the combination of all the various components of the pharmaceutical composition, the various structural units, functionalization of structural units (with various moieties), levels of crosslinking (if crosslinked), molecular weight, concentrations, or other chemical features of the disclosed compositions contribute to the tunability of the properties of the compositions disclosed herein. In several embodiments, by changing one or more of these features, one or more properties of the compositions can be tuned according to the preferred properties described herein. In several embodiments, the NO release rate, antimicrobial effect, water solubility, degradation rate, viscosity, gel firmness (where the scaffold forms a gel), viscoelasticity, modulus, etc. are tunable.

In several embodiments, properties of the polymer and or composition prepared therefrom can be tuned by adjusting the molecular weight of certain polymers used in the formulation. In several embodiments, the weight-average molecular weight (MW) in kDa of polymers disclosed herein are greater than or equal to about: 2.5, 5.0, 7.0, 10, 15, 30, 50, 100, 200, 500, 750, 1,000, 2,000, 10,000, or ranges including and/or spanning the aforementioned values. In several embodiments, the number-average molecular weight (M_{W}) in kDa of polymers disclosed herein are greater than or equal to about: 2.5, 5.0, 7.0, 10, 15, 30, 50, 90, 100, 200, 500, 700, 1,000, 2,000, 10,000, or ranges including and/or spanning the aforementioned values. In several embodiments, the polymers disclosed herein may have n repeat units. In several embodiments, n equal to or at least about: 10, 25, 50, 100, 250, 500, 1000, 2500, 5000, 10000, or ranges including and/or spanning the aforementioned values. In several embodiments, size exclusion chromatography (SEC) can be used to measure the molecular weight of the scaffold structures disclosed herein. In several embodiments, multi-angle light scattering (SEC-MALS) detectors can be used. In several embodiments, the scaffold structures can be characterized using their polydispersity index. The polydispersity index (PDI) is a measure of the distribution of molecular mass in a given polymer sample. PDI can be calculated by dividing the weight average molecular weight and the number average molecular weight. In several embodiments, the scaffold structures have a PDI of greater than or equal to about: 1.05, 1.1, 1.2, 1.3, 1.5, 1.7, 1.8, 1.9, 2.0, or ranges including and/or spanning the aforementioned values.

In several embodiments, the compositions (including all the components) may be water soluble and/or mutually miscible. In several embodiments, the compositions are soluble in water (at about 20° C.) at a concentration of greater than or equal to about: 1 mg/ml, 10 mg/ml, 20 mg/ml, 50 mg/ml, 100 mg/ml, 200 mg/ml, 300 mg/ml, 400 mg/ml, 500 mg/ml, or ranges including and/or spanning the aforementioned values.

According to several embodiments, the NO donor can be formulated within a pharmaceutical formulation at a concentration equal to or at least about: 100 μg/mL, and can be higher, e.g. about 1 mg/ml, about 5 mg/ml, about 10 mg/ml, about 20 mg/ml, 25 mg/ml, 50 mg/ml, 75 mg/ml, 100 mg/ml or about 200 mg/ml or higher. In illustrative embodiments, a polymeric species can be formulated within a pharmaceutical formulation at a concentration equal to or at least about: 100 μg/mL, and can be higher, e.g. about 1 mg/ml, about 5 mg/ml, about 10 mg/ml, about 20 mg/ml, 25 mg/ml, 50 mg/ml, 75 mg/ml, 100 mg/ml or about 200 mg/ml or higher. The amount of the polymer in the aqueous composition can be at least about 10% by weight, based on the weight of the NO donor, and may be higher, e.g., at least about 20% by weight, at least about 30% by weight, or at least about 50% by weight, same basis. Any combinations of NO donors and polymers in an aqueous composition are selected to be mutually miscible. As noted above, the NO donor with antimicrobial activity and the polymer are considered mutually miscible if at least about 90% of the polymeric components remain mutually soluble 24 hours after mixing and maintaining at room temperature in water at a concentration of each polymer of 1 mg/ml, upon visible examination. Surprisingly, such mutual miscibility of the water soluble polymers with the NO donors can be achieved, despite an expectation of phase separation at the 1 mg/ml concentrations and molecular weights described herein. The aqueous compositions described herein can be prepared by intermixing the individual formulation components with water, e.g., at room temperature with stirring.

In several embodiments, the composition disclosed herein have properties characteristic of a viscous fluid and/or of a gel. In several embodiments, a composition has a gelling point at room temperature (in water or PBS) at a concentration (in w/w %) of less than or equal to about: 0.5%, 1%, 2.5%, 5%, 10%, or ranges including and/or spanning the aforementioned values. In several embodiments, the composition may have a gelling point in water. In several embodiments, the composition gels in water (at about 20° C.) at a concentration of greater than or equal to about: 0.5 mg/ml, 1 mg/ml, 10 mg/ml, 20 mg/ml, 50 mg/ml, 100 mg/ml, 250 mg/ml, or ranges including and/or spanning the aforementioned values. In several embodiments, at a concentration of 5% w/w solution, the polymers have a viscosity (in cPa·s at 20° C.) of equal to or at least about: 10, 50, 100, 1,000, 2,000, 5,000, 10,000, or ranges including and/or spanning the aforementioned values. In several embodiments, the polymers have an intrinsic viscosity of equal to or greater than about: 0.5 m³/kg, 1.0 m³/kg, 2.0 m³/kg, 4.0 m³/kg, 8.0 m³/kg, or ranges including and/or spanning the aforementioned values.

In several embodiments, at a concentration of 5% w/w solution, the compositions have a firmness of equal to or at least about: 1.0 mN, 2.5 mN, 5 mN, 10 mN, 15 mN, 20 mN, 30 mN, 50 mN, or ranges including and/or spanning the aforementioned values. In several embodiments, at a concentration of 5% w/w solution, the formulations have a work of adhesion (in mN*mm) of equal to or at least about: 1.0, 2.5, 5, 10, 15, 20, 30, 50, 100, or ranges including and/or spanning the aforementioned values. In several embodiments, at a concentration of 5% w/w solution, the compositions have a storage modulus (G′) in Pa of equal to or at least about: 250, 500, 1,000, 2,000, 4,000, 5,000, 10,000, or ranges including and/or spanning the aforementioned values. In several embodiments, at a concentration of 5% w/w solution, the compositions have an elastic modulus (G″) in Pa of equal to or at least about: 25, 50, 100, 200, 400, 500, 1,000, 2,000, 5,000, 10,000, or ranges including and/or spanning the aforementioned values. In several embodiments, the aqueous composition is characterized by a barrier activity, as measured by a decrease in the diffusion rate of an anionic dye of more than 2 logs at a total scaffold concentration of 40 mg/ml or less.

In several embodiments, the formulation is a gel and the gel are stable at a variety of temperatures 20° C. (e.g., 400 C, 45° C., 550 C, 60° C., 80° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year or greater)).

In several embodiments, the viscosity of the composition increases with increasing temperature, as described above. In several embodiments, the viscosity of the composition decreases with decreasing temperature. For example, if the composition is above the gelling temperature, then the composition has a relatively high viscosity, such as in the form of a gel. In several embodiments, if the composition is cooled to below the gelling temperature, then the composition decreases in viscosity, such as in the form of a liquid. In several embodiments, as such, the polymers as disclosed herein may be reversible polymers (e.g., thermoreversible polymers), where the transition from liquid to gel may be reversed upon exposure to appropriate conditions. For instance, as described above, compositions of the present disclosure include thermoreversible polymers, where the viscosity of the composition may be changed depending on the temperature of the composition. In several embodiments, the tunability of the viscosity enables a tailored composition profile upon delivery (e.g., more liquid at a delivery temperature and more viscous at, for example, body temperature).

In several embodiments, the compositions are characterized by a degree of swelling when exposed to water. In some embodiments, the swelling degree % of the composition disclosed herein is equal to or at least about: 100, 250, 500, 1,000, 2,000, 5,000, or ranges including and/or spanning the aforementioned values. In other words, the composition may swell or otherwise expand by 2×, 4×, 5×, 10×, 20×, 50×, 100×, or more.

In certain embodiments, the compositions disclosed herein have a gelling temperature similar to the normal body temperature of a subject, such as similar to human body temperature, or 37° C. By gelling temperature is meant the point on intersection between the plot for the elastic modulus and the plot for the viscous modulus. In some cases, if the composition is below the gelling temperature, then the composition has a relatively low viscosity, such as in the form of a liquid. In some instances, if the composition is above the gelling temperature, then the composition increases in viscosity (e.g., polymerizes), such that the composition is in the form of a gel. Compositions that transition from a liquid to a gel may facilitate administration of the composition to the subject, for example by facilitating injection of a low viscosity (e.g., liquid) composition at a temperature below the gelling temperature. After injection of the composition to the target treatment site, the temperature of the composition may increase due to absorption of heat from the surrounding body tissue, such that the composition increases in viscosity (e.g., transitions from a liquid to a gel, or polymerizes), thus providing structural and/or geometric support to the body tissue at the target treatment site. In some instances, gelling of the composition at the target treatment site may also facilitate retention of the composition at the treatment site by reducing the diffusion and/or migration of the composition away from the treatment site. In certain embodiments, the composition has a gelling temperature of 30° C. to 40° C., such as from 32° C. to 40° C., including from 35° C. to 40° C. In certain instances, the composition has a gelling temperature of 37° C.

In some embodiments, the methods disclosed herein provide NO-releasing polymers having NO storage capacities (in μmol NO/mg polymers) of greater than or equal to about: 0.25, 0.4, 0.5, 1.0, 1.5, 2.0, 3.0, or ranges including and/or spanning the aforementioned values. In some embodiments, within 2 h of being added to a PBS buffer solution as described in the Examples, the NO-releasing polymers, release greater than or equal to about: 25%, 50%, 75%, 85%, 90%, 95%, 100%, or ranges including and/or spanning the aforementioned values, their total wt % of bound NO. In several embodiments, NO release in use for reducing or eliminating a biofilm occurs in similar amounts, e.g., about 20-25%, about 30-50%, about 60-75%, at least 80%, at least 85%, at least 90%, at least 95%, ranges including and/or spanning the aforementioned values, of the total wt % of bound NO.

In some embodiments, the NO release may occur over a period of about 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In several embodiments, the NO release half-life is equal to or at least about: 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, 60 hours, or ranges including and/or spanning the aforementioned values. In some embodiments, nitrosamine is not present during NO release. As used herein the phrase “nitrosamine is not present” refers broadly to levels of nitrosamine which are not detectable as determined by a UV-vis spectrum (or by other accepted methods in the art).

In some embodiments, the disclosed scaffolds and/or polymers of the disclosed compositions have a degradation rate per hour in an amylase enzyme exposure assay of less than or equal to about: 0.2%, 0.5%, 1.0%, 1.5%, 2.5%, 5.0%, 10%, or ranges including and/or spanning the aforementioned values.

In some embodiments, the disclosed functionalized NO-releasing polymers have antimicrobial activity. In some embodiments, the disclosed functionalized NO-releasing polymers provide greater than or equal to 90% bacterial reduction in a bacterial viability assay performed under static conditions over 2 hours against one or more of P. aeruginosa, S. aureus P. gingivalis, A. actinomycetemcomitans, A. viscosus, and/or S. mutans at a polymer concentration of equal to or less than about: 8 mg/ml, 6 mg/ml, 4 mg/ml, 2 mg/ml, 1 mg/ml, 0.5 mg/ml, or ranges including and/or spanning the aforementioned values. In some embodiments, the disclosed functionalized NO-releasing polymers provide greater than or equal to 99% bacterial reduction and/or a 2 to 3 log reduction in a bacterial viability assay performed under static conditions over 2 hours against a gram positive bacteria at a polymer concentration of equal to or less than about: 8 mg/ml, 6 mg/ml, 4 mg/ml, 2 mg/ml, 1 mg/ml, 0.5 mg/ml, or ranges including and/or spanning the aforementioned values. In some embodiments, the disclosed functionalized NO-releasing polymers provide greater than or equal to 99% bacterial reduction and/or a 2 to 3 log reduction in a bacterial viability assay performed under static conditions over 2 hours against a gram negative bacteria at a polymer concentration of equal to or less than about: 8 mg/ml, 6 mg/ml, 4 mg/ml, 2 mg/ml, 1 mg/ml, 0.5 mg/ml, or ranges including and/or spanning the aforementioned values. In several embodiments, bacterial reduction is greater than 95%, greater than 98%, or greater than 99%.

### Combination Therapy for Particular Use in Treating Covid-19 Infections

The compounds described herein can be combined with additional compounds useful for treating the disease states also treated by the release of NO. In particular, the compounds discussed below can be used in combination therapy to treat Covid-19 infections, or other respiratory infections with similar pathology.

Various compounds that can be combined with the compounds described herein are discussed below.

In one aspect of this embodiment, the at least one other active agent is selected from the group consisting of fusion inhibitors, entry inhibitors, protease inhibitors, polymerase inhibitors, antiviral nucleosides, such as remdesivir, GS-441524, N4-hydroxycytidine, and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

Umifenovir (also known as Arbidol) is a representative fusion inhibitor.

Representative entry inhibitors include Camostat, luteolin, MDL28170, SSAA09E2, SSAA09E1 (which acts as a cathepsin L inhibitor), SSAA09E3, and tetra-O-galloyl-β-D-glucose (TGG). The chemical formulae of certain of these compounds are provided below:

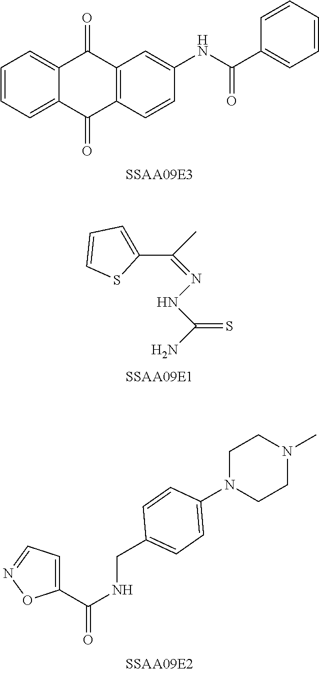 [SMILES: O=C(Nc1ccc2c(c1)C(=O)c1ccccc1C2=O)c1ccccc1 ; C/C(=N/NC(N)=S)c1cccs1 ; CN1CCN(c2ccc(CNC(=O)c3ccno3)cc2)CC1]

Other entry inhibitors include the following:

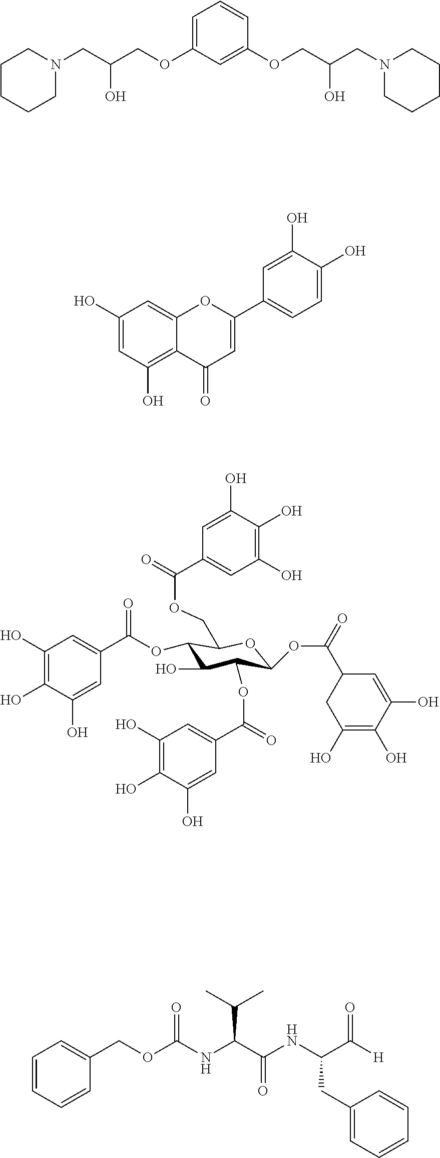

Remdesivir, Sofosbuvir, ribavirin, JDX-184 and GS-441524 have the following formulas:

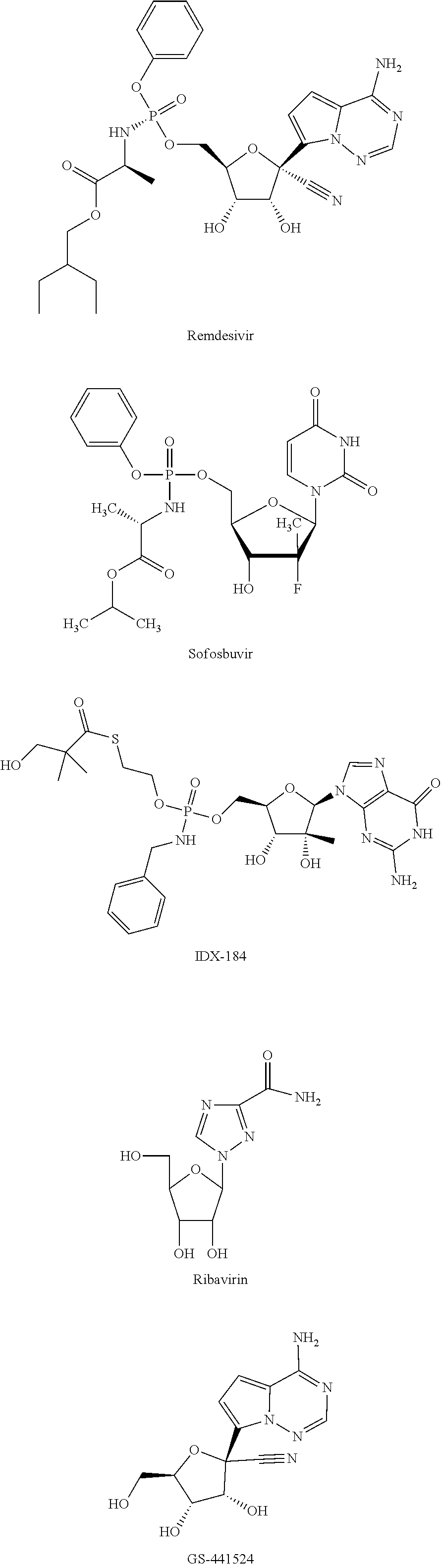

Additionally, one can administer compounds which inhibit the cytokine storm, anti-coagulants and/or platelet aggregation inhibitors that address blood clots, or compounds which chelate iron ions released from hemoglobin by viruses such as COVID-19.

Representative ACE-2 inhibitors include sulfhydryl-containing agents, such as alacepril, captopril (capoten), and zefnopril, dicarboxylate-containing agents, such as enalapril (vasotec), ramipril (altace), quinapril (accupril), perindopril (coversyl), lisinopril (listril), benazepril (lotensin), imidapril (tanatril), trandolapril (mavik), and cilazapril (inhibace), and phosphonate-containing agents, such as fosinopril (fositen/monopril).

For example, when used to treat or prevent infection, the active compound or its prodrug or pharmaceutically acceptable salt can be administered in combination or alternation with another antiviral agent including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

A number of agents for combination with the compounds described herein are disclosed in Ghosh et al., “Drug Development and Medicinal Chemistry Efforts Toward SARS-Coronavirus and Covid-19 Therapeutics,” ChemMedChem 10.1002/cmdc.202000223.

Nonlimiting examples of antiviral agents that can be used in combination with the compounds disclosed herein include those listed below.

### Compounds for Inhibiting the Cytokine Storm

Throughout its activation, the inflammatory response must be regulated to prevent a damaging systemic inflammation, also known as a “cytokine storm.” A number of cytokines with anti-inflammatory properties are responsible for this, such as IL-10 and transforming growth factor R (TGF-β). Each cytokine acts on a different part of the inflammatory response. For example, products of the Th2 immune response suppress the Th1 immune response and vice versa.

By resolving inflammation, one can minimize collateral damage to surrounding cells, with little or no long-term damage to the patient. Accordingly, in addition to using the compounds described herein to inhibit the viral infection, one or more compounds which inhibit the cytokine storm can be co-administered.

Compounds which inhibit the cytokine storm include compounds that target fundamental immune pathways, such as the chemokine network and the cholinergic anti-inflammatory pathway.

JAK inhibitors, such as JAK 1 and JAK 2 inhibitors, can inhibit the cytokine storm, and in some cases, are also antiviral. Representative JAK inhibitors include those disclosed in U.S. Pat. No. 10,022,378, such as Jakafi, Tofacitinib, and Baricitinib, as well as LY3009104/INCB28050, Pacritinib/SB1518, VX-509, GLPG0634, INC424, R-348, CYT387, TG 10138, AEG 3482, and pharmaceutically acceptable salts and prodrugs thereof.

HMGB1 antibodies and COX-2 inhibitors can be used, which downregulate the cytokine storm. Examples of such compounds include Actemra (Roche). Celebrex (celecoxib), a COX-2 inhibitor, can be used. IL-8 (CXCL8) inhibitors can also be used.

Chemokine receptor CCR2 antagonists, such as PF-04178903 can reduce pulmonary immune pathology.

Selective α7Ach receptor agonists, such as GTS-21 (DMXB-A) and CNI-1495, can be used. These compounds reduce TNF-α. The late mediator of sepsis, HMGB1, downregulates IFN-7 pathways, and prevents the LPS-induced suppression of IL-10 and STAT 3 mechanisms.

### Compounds for Treating or Preventing Blood Clots

Viruses that cause respiratory infections, including Coronaviruses such as Covid-19, can be associated with pulmonary blood clots, and blood clots that can also do damage to the heart.

The compounds described herein can be co-administered with compounds that inhibit blood clot formation, such as blood thinners, or compounds that break up existing blood clots, such as tissue plasminogen activator (TPA), Integrilin (eptifibatide), abciximab (ReoPro) or tirofiban (Aggrastat).

Blood thinners prevent blood clots from forming, and keep existing blood clots from getting larger. There are two main types of blood thinners. Anticoagulants, such as heparin or warfarin (also called Coumadin), slow down biological processes for producing clots, and antiplatelet aggregation drugs, such as Plavix, aspirin, prevent blood cells called platelets from clumping together to form a clot.

By way of example, Integrilin® is typically administered at a dosage of 180 mcg/kg intravenous bolus administered as soon as possible following diagnosis, with 2 mcg/kg/min continuous infusion (following the initial bolus) for up to 96 hours of therapy.

Representative platelet aggregation inhibitors include glycoprotein IIB/IIIA inhibitors, phosphodiesterase inhibitors, adenosine reuptake inhibitors, and adenosine diphosphate (ADP) receptor inhibitors. These can optionally be administered in combination with an anticoagulant.

Representative anti-coagulants include coumarins (vitamin K antagonists), heparin and derivatives thereof, including unfractionated heparin (UFH), low molecular weight heparin (LMWH), and ultra-low-molecular weight heparin (ULMWH), synthetic pentasaccharide inhibitors of factor Xa, including Fondaparinux, Idraparinux, and Idrabiotaparinux, directly acting oral anticoagulants (DAOCs), such as dabigatran, rivaroxaban, apixaban, edoxaban and betrixaban, and antithrombin protein therapeutics/thrombin inhibitors, such as bivalent drugs hirudin, lepirudin, and bivalirudin and monovalent argatroban.

Representative platelet aggregation inhibitors include pravastatin, Plavix (clopidogrel bisulfate), Pletal (cilostazol), Effient (prasugrel), Aggrenox (aspirin and dipyridamole), Brilinta (ticagrelor), caplacizumab, Kengreal (cangrelor), Persantine (dipyridamole), Ticlid (ticlopidine), Yosprala (aspirin and omeprazole).

Additional Compounds that can be Used

Additional compounds and compound classes that can be used in combination therapy include the following: Antibodies, including monoclonal antibodies (mAb), Arbidol (umifenovir), Actemra (tocilizumab), APNO1 (Aperion Biologics), ARMS-1 (which includes Cetylpyridinium chloride (CPC)), ASC09 (Ascletis Pharma), AT-001 (Applied Therapeutics Inc.) and other aldose reductase inhibitors (ARI), ATYR1923 (aTyr Pharma, Inc.), Aviptadil (Relief Therapeutics), Azvudine, Bemcentinib, BLD-2660 (Blade Therapeutics), Bevacizumab, Brensocatib, Calquence (acalabrutinib), Camostat mesylate (a TMPRSS2 inhibitor), Camrelizumab, CAP-1002 (Capricor Therapeutics), CD24Fcm, Clevudine, (OncoImmune), CM4620-IE (CalciMedica Inc., CRAC channel inhibitor), Colchicine, convalescent plasma, CYNK-001 (Sorrento Therapeutics), DAS181 (Ansun Pharma), Desferal, Dipyridamole (Persantine), Dociparstat sodium (DSTAT), Duvelisib, Eculizumab, EIDD-2801 (Ridgeback Biotherapeutics), Emapalumab, Fadraciclib (CYC065) and seliciclib (roscovitine) (Cyclin-dependent kinase (CDK) inhibitors), Farxiga (dapagliflozin), Favilavir/Favipiravir/T-705/Avigan, Galidesivir, Ganovo (danoprevir), Gilenya (fingolimod) (sphingosine 1-phosphate receptor modulator), Gimsilumab, IFX-1, Ilaris (canakinumab), intravenous immunoglobulin, Ivermectin (importin a/P inhibitor), Kaletra/Aluvia (lopinavir/ritonavir), Kevzara (sarilumab), Kineret (anakinra), LAU-7b (fenretinide), Lenzilumab, Leronlimab (PRO 140), LY3127804 (an anti-Ang2 antibody), Leukine (sargramostim, a granulocyte macrophage colony stimulating factor), Losartan, Valsartan, and Telmisartan (Angiotensin II receptor antagonists), Meplazumab, Metablok (LSALT peptide, a DPEP1 inhibitor), Methylprednisolone and other corticosteroids, MN-166 (ibudilast, Macrophage migration inhibitory factor (MIF) inhibitor), MRx-4DP0004 (a strain of Bifidobacterium breve, 4D Pharma), Nafamostat (a serine protease inhibitor), Neuraminidase inhibitors like Tamiflu (oseltamivir), Nitazoxanide (nucleocapsid (N) protein inhibitor), Nivolumab, OT-101 (Mateon), Novaferon (man-made Interferon), Opaganib (yeliva) (Sphingosine kinase-2 inhibitor), Otilimab, PD-1 blocking antibody, peginterferons, such as peginterferon lambda, Pepcid (famotidine), Piclidenoson (A3 adenosine receptor agonist), Prezcobix (darunavir), PUL-042 (Pulmotect, Inc., toll-like receptor (TLR) binder), Rebif (interferon beta-la), RHB-107 (upamostat) (serine protease inhibitor, RedHill Biopharma Ltd.), Selinexor (selective inhibitor of nuclear export (SINE)), SNG001 (Synairgen, inhaled interferon beta-la), Solnatide, stem cells, including mesenchymal stem cells, MultiStem (Athersys), and PLX (Pluristem Therapeutics), Sylvant (siltuximab), Thymosin, TJM2 (TJ003234), Tradipitant (neurokinin-1 receptor antagonist), Truvada (emtricitabine and tenofovir), Ultomiris (ravulizumab-cwvz), Vazegepant (CGRP receptor antagonist or blocker), and Xofluza (baloxavir marboxil).

### Repurposed Antiviral Agents

A number of pharmaceutical agents, including agents active against other viruses, have been evaluated against Covid-19, and found to have activity. Any of these compounds can be combined with the compounds described herein. Representative compounds include lopinavir, ritonavir, niclosamide, promazine, PNU, UC2, cinanserin (SQ 10,643), Calmidazolium (C3930), tannic acid, 3-isotheaflavin-3-gallate, theaflavin-3,3′-digallate, glycyrrhizin, S-nitroso-N-acetylpenicillamine, nelfinavir, niclosamide, chloroquine, hydroxychloroquine, 5-benzyloxygramine, ribavirin, Interferons, such as Interferon (IFN)-α, IFN-β, and pegylated versions thereof, as well as combinations of these compounds with ribavirin, chlorpromazine hydrochloride, triflupromazine hydrochloride, gemcitabine, imatinib mesylate, dasatinib, and imatinib.

### V. Methods of Treatment

An unmet need in the area of wound healing, general surgery, and orthopedic surgery is for a antimicrobial material that can form a gel, that can release NO at a requisite rate, and that can degrade during a desired timeframe. This tailored degradation rate can be made to comport with the healing cycle of each specific condition and/or can comport to a time where the wound is at high risk of infection. Examples of these conditions include procedures such as hernia repair, diabetic foot ulcer healing, and orthopedic tendon repairs to name only a few. In several embodiments, the compositions disclosed herein are targeted towards compositions that have tailorable degradation times.

Some embodiments provide a method for treating a tissue defect comprising positioning any of the compounds described herein at, over, or into the tissue defect. In several embodiments, the tissue defect is a wound. Several embodiments provide a method for treating a wound, for performing tissue repair, and/or for providing tissue and organ supplementation. In several embodiments, the first step of treating a tissue defect, wound, and/or supplementing and replacing tissue involves identifying a patient in need of an antimicrobial scaffold to aid in the remedying and healing of a tissue defect, healing of a wound, or in need of a tissue supplement.

A non-limiting list of patients in need of an antimicrobial scaffold includes patients suffering tissue defects. In several embodiments, the patients in need of an antimicrobial scaffold suffer from wounds including those from burns, skin ulcers, lacerations, bullet holes, animal bites, and other wounds prone to infection. Antimicrobial compounds can also be used to treatdiabetic foot ulcers, venous leg ulcers, pressure ulcers, amputation sites, in other skin trauma, or in the treatment of other wounds or ailments. Patients in need of an antimicrobial scaffold also include patients in need of repair and supplementation of tendons, ligaments, fascia, and dura mater. The compounds can also be used in supplement tissue in procedures including, but not limited to, rotator cuff repair, Achilles tendon repair, leg or arm tendon or ligament repair (e.g., torn ACL), vaginal prolapse repair, bladder slings for urinary incontinence, breast reconstruction following surgery, hernia repair, staple or suture line reinforcement, bariatric surgery repair, pelvic floor reconstruction, dural repair, gum repair, bone grafting, and reconstruction. Further, a patient in need of an antimicrobial scaffold also includes one in need of tissue or organ replacement. In several embodiments, the compositions described herein can be used as fillers and/or to supplement and/or replace tissue by acting as an artificial extracellular matrix. In such an application, an antimicrobial scaffold can be used to support cell and tissue growth. Briefly, cells can be taken from a patient or a viable host and seeded on an antimicrobial scaffold either in vivo or ex vivo. Then as the patient's natural tissues invade the material, it is tailored to degrade and leave only naturally occurring tissues and cells free of bacterial infection.

In several embodiments, applications also include delivery of therapeutic molecules to a localized site, use as adhesives or sealants, and as viscosupplements, and in wound healing, among others. The stabilized compositions may also be used as tissue fillers, dermal fillers, bone fillers, bulking agents, e.g., as a urethral or an esophageal bulking agent, and embolic agents as well as agents to repair cartilage defects/injuries and agents to enhance bone repair and/or growth. In several embodiments, a composition comprising an antimicrobial scaffold can be placed in or on a patient in, for example, a void space to fill the space.

In several embodiments, the compounds are used to repair injured tissue. In several embodiments, the composition is formulated for administration to a target treatment site in a subject. For example, the composition may be formulated to facilitate administration to a damaged or infected tissue in a subject.

In several embodiments, after administration of the composition (e.g., gallium and NO donor, which comprises an antimicrobial scaffold), the composition may increase in temperature due to absorption of heat from surrounding body tissue of the subject. In several embodiments, the body temperature of the subject is sufficient to cause the composition to increase in viscosity (e.g., transition from a liquid to a gel. In several embodiments, the increase in viscosity (e.g., gelling) may give rise to a 3-dimensional network sufficient to provide structural and/or geometric support to a body tissue, such as a cardiac tissue (e.g., a cardiac tissue of an infarct region). In several embodiments, a syringe or catheter may be used to inject the composition in vivo. In several embodiments, the composition may be injected directly to the treatment site, or may be allowed to partially pre-heat in the syringe in order to increase the viscosity of the composition prior to injection. In several embodiments, a pre-heated formulation may reduce the possibility that a less viscous composition may diffuse and/or migrate away from the tissue area of interest after injection.

Dental caries (e.g., tooth decay) is another important disease state that affects 60%-70% school age children and the majority of adults in most industrialized countries. Worldwide, 11% of the total population suffers from severe periodontitis, which contributes to tooth loss and systematic diseases such as coronary, cardiovascular, stroke, and adverse pregnancy outcomes. Of >700 microorganisms in the oral cavity, cariogenic bacteria (e.g., Streptococcus mutans, Actinomyces viscosus) and periodontal pathogens (e.g., Porphyromonas gingivalis, Aggregatibacter actinomycetemcomitans) play a major role in the initiation and progression of oral diseases. Oral disease is among the most prevalent health problems faced by humans. Gram-positive cariogenic (e.g., Streptococcus mutans, Actinomyces viscosus) and Gram-negative periodontal (e.g., Porphyromonas gingivalis, Aggregatibacter actinomycetemcomitans) bacteria represent the main aggravators associated with the evolution and progression of dental caries and periodontal disease, respectively. Unfortunately, current treatments to combat these pathogens come with undesirable side effects. For example, the systemic use of antibiotics may result in gastrointestinal disturbance and foster bacterial resistance. Chlorhexidine, a common oral antiseptic, can alter taste, stain teeth and tongue, and irritate buccal mucosa. Macromolecule NO-delivering vehicles (e.g., silica nanoparticles, gold, etc.) kill Gram-negative periodontal pathogens. However, these materials have not been demonstrated to kill Gram-positive cariogenic bacteria at a safe concentration (e.g., a concentration that is bacteriocidal but non-toxic towards mammalian cells). Similar with those nanomaterials, the lack of biodegradability and potential cytotoxicity of the silica nanoparticles also hinders their future for biomedical application. Current research also focuses on utilizing nanomaterials including silver, gold, zinc, and copper, as replacement for traditional antibiotics that suffered from fostering bacterial resistance. However, these nanomaterials may accumulate inside the body and may cause accumulative toxicity, limiting their future for certain applications. Developing oral therapeutics that are capable of killing those disease-causing bacteria is important to maintain a healthy oral cavity. In several embodiments, the compositions disclosed herein (including, for example, NO scaffolds), resolve one or more of these issues or others.

The compositions described herein may be used in patients with cystic fibrosis, bronchiectasis, chronic obstructive pulmonary diseases (COPD), or in the patients on ventilators. These patients are also susceptible to biofilm forming infections, which the compositions described herein are effective against.

In several embodiments, the compositions disclosed herein may be used as eye drop formulations (e.g., artificial tears). In several embodiments, the composition comprises from about 0.1% to about 1.0% of the scaffold (or at a concentration as disclosed elsewhere herein). In several embodiments, the mixture comprises more than one type of antimicrobial compound.

Cystic fibrosis (CF) is a genetic disorder characterized by poor mucociliary clearance and chronic bacterial infections. As shown herein, in several embodiments, nitric oxide (NO) has broad spectrum antibacterial activity against CF-relevant bacteria, making it an attractive alternative to traditional antibiotics. Treatment with NO limits bacterial resistance due to its multiple biocidal mechanisms (e.g., induction of nitrosative and oxidative stress). It has surprisingly been found that by storing NO on a scaffold using one of the disclosed designs, bactericidal efficacy is improved and systemic cytotoxicity is reduced. Treatments are effective against planktonic and biofilm-based pathogens, and cytotoxicity assays against mammalian lung cells demonstrate little harm to a treated subject's cells. It has also surprisingly been found that the combination of gallium and a NO donor is synergistically effective.

Cystic Fibrosis (CF) is a debilitating disease characterized by chronic bacterial infection of the lungs, resulting in life expectancies as low as two decades. A genetic defect in the CF transmembrane conductance regulator (CFTR) impedes the normal transport of ions (e.g., Cl—) to the airway surface liquid, inhibiting water transport. As such, the airway epithelium dehydrates, creating thickened mucus that can no longer be efficiently cleared via mucociliary clearance mechanisms. As goblet cells continually excrete mucins into the dehydrated airway, mucus accumulation is accelerated to the point where the cilia become damaged, or nonfunctional, and are unable to clear mucus from the airway. Planktonic bacteria thrive in this static environment, promoting the formation of complex communities of pathogenic bacteria known as biofilms. The exopolysaccharide matrix produced by these biofilms inhibits oxygen diffusion, creating pockets of anaerobic environments and altering bacterial metabolism. This combination of a concentrated mucus layer and robust biofilms severely decreases the antibacterial efficacy of common CF therapies.

In several embodiments, the microbial load to be reduced and/or eliminated comprises drug-resistant bacteria. In several embodiments, the drug-resistant bacteria comprise carbapenem-resistant Enterobacteriaceae. In several embodiments, the drug-resistant bacteria comprise Methicillin-resistant Staphylococcus aureus. In several embodiments, the microbe comprises human immunodeficiency virus, herpes simplex virus, papilloma virus, parainfluenza virus, influenza, hepatitis, Coxsackie Virus, herpes zoster, measles, mumps, rubella, rabies, pneumonia, (hemorrhagic viral fevers, H1N1, and the like), prions, parasites, fungi, mold, yeast and bacteria (both gram-positive and gram-negative) including, among others, Candida albicans, Aspergillus niger, Escherichia coli (E. coli), Pseudomonas aeruginosa (P. aeruginosa), and Staphylococcus aureus (S. aureus), Group A streptococci, S. pneumoniae, Mycobacterium tuberculosis, Campylobacter jejuni, Salmonella, Shigella, P. gingivalis, A. actinomycetemcomitans, A. viscosus, and/or S. mutans and a variety of drug resistant bacteria. The terms microorganism and microbe shall be used interchangeably. Microbes can include wild-type, genetically-engineered or modified organisms. In several embodiments, the formulations and methods disclosed herein are for topical use or treatment of a surface, such as the oral mucosa.

In some embodiments, the compositions of the present disclosure thereof may be administered by direct injection or application to, for example, an injured tissue. Suitable routes also include injection or application to a site adjacent to the injured tissue. Administration may include parenteral administration (e.g., intravenous, intramuscular, or intraperitoneal injection), subcutaneous administration, administration into vascular spaces, and/or administration into joints (e.g., intra-articular injection). Additional routes of administration include intranasal, topical, vaginal, rectal, intrathecal, intraarterial, and intraocular routes. In several embodiments, the compositions disclosed herein can be applied as a gel to a site of treatment. In several embodiments, the compositions can be applied as a liquid.

In several embodiments, liquid preparations for oral or topical administration can take the form of, for example, solutions, syrups or suspensions, or they can be presented as a dry product for constitution with water or another suitable vehicle before use. Such liquid preparations can be prepared by conventional techniques with pharmaceutically acceptable additives, such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations also can contain buffer salts, flavoring, coloring and sweetening agents as appropriate. Preparations for oral administration can be suitably formulated to give controlled release of the active compound. For buccal administration the compositions can take the form of tablets or lozenges formulated in a conventional manner.

In several embodiments, the disclosed compositions also can be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt). The compositions also can be formulated in rectal compositions (e.g., suppositories or retention enemas containing conventional suppository bases, such as cocoa butter or other glycerides), creams or lotions, or transdermal patches.

Pharmaceutical formulations also are provided which are suitable for administration as an aerosol by inhalation. In several embodiments, the compounds described herein are formulated in solution and/or aerosol form. In several embodiments, the desired formulation can be placed in a small chamber and nebulized. Nebulization can be accomplished by compressed air or by ultrasonic energy to form a plurality of liquid droplets or solid particles comprising the NO-releasing compounds. For example, the presently disclosed compositions comprising the compounds described herein can be administered via inhalation to treat bacterial infections related to cystic fibrosis. Cystic fibrosis-related bacterial infections include, but are not limited to stenotrophomonis, mybacterium avium intracellulaire and m. abcessus, burkhoderia cepacia and Pseudomonas aeruginosa (P. aeruginosa) infections.

Synergy with Gallium

Microbial infections pose a great challenge to human health in community and hospital settings. Several chronic infections, such as those associated with implanted devices, chronic wounds, and cystic fibrosis, are frequently caused by biofilm-forming pathogens such as Pseudomonas aeruginosa and Staphylococcus aureus. Biofilms are cooperative communities of bacteria encapsulated by an exopolysaccharide (EPS) matrix protecting the bacteria from host immune response and antibiotics. It has been reported that eradication of biofilms may require up to 1000 times higher antibiotic concentrations relative to those needed for plankton bacteria. Resistant respiratory infections are particularly difficult to treat because they form protective biofilms inside airway mucus and can survive for decades. There exists a need in the art for new antibacterial compositions because of the resistance of biofilms to conventional antibacterial agents.

Gallium has been reported to possess both antimicrobial and immunosuppressant effects. Activity towards viruses (Narayanasamy, P., Switzer, B. & Britigan, B. Prolonged-acting, Multi-targeting Gallium Nanoparticles Potently Inhibit Growth of Both HIV and Mycobacteria in Co-Infected Human Macrophages. Sci Rep 5, 8824 (2015) has been demonstrated. Likewise, gallium has been presented as damaging key iron-dependent enzymes in bacteria (See Goss C H, Kaneko Y, Khuu L, et al. Gallium disrupts bacterial iron metabolism and has therapeutic effects in mice and humans with lung infections. Sci Transl Med. 2018; 10(460):eaat7520. This observation has led to the development of gallium citrate as a pharmaceutical product (AR-501 by Aridis). Gallium has been studied against planktonic, biofilm, and in vivo PA (See Kaneko Y, Thoendel M, Olakanmi 0, Britigan B E, Singh P K and J Clin Invest. 2007; 117(4):877-888). Tested against planktonic and macrophage-grown non-tuberculosis mycobacteria (NTM), gallium has shown some promise for the treatment of chronic infections (See Abdalla M Y, Switzer B L, Goss C H, Aitken M L, Singh P K, Britigan B E and Antimicrob Agents Chemother. 2015; 59(8):4826-4834

The inventors surprising discovered that the combination of gallium and a nitric-oxide donor produced a synergistic anti-microbial effect. This combination of gallium and a nitric-oxide releasing compound was surprisingly effective against biofilms, which are notoriously difficult to treat with antimicrobial composition. It is also expected that compositions comprising gallium, at least one nitric oxide donor, and at least one siderophore also exhibit synergistic effects against plankton bacteria and biofilms.

Several embodiments disclosed herein provide the synthesis and characterization of N-diazeniumdiolate NO donor-modified scaffolds and their use in antimicrobial applications. In several embodiments, the scaffolds comprise a modified cyclodextrin, such as disclosed in US Patent Application 2019/0343869. In several embodiments, the scaffolds comprise biopolymers. In several embodiments, the scaffolds comprise biocompatible polymers. In several embodiments, the scaffolds comprise one or more saccharide units and/or are polysaccharides. In several embodiments, the scaffolds comprise one or more chitosan, hyaluronic acid (HA), carboxymethylcellulose (CMC), hydroxyethyl cellulose, methyl cellulose, cellulose, alginate (including 1,4-linked α-l-guluronic acid (G) and β-d-mannuronic acid (M) units), collagen, gelatin, cyclodextrin (e.g., having 5 (α), 6 (β), 7 (γ), or more α-D-glucopyranosides), aminoglycosides (e.g., kanamycin, streptomycin, tobramycin, gentamicin, neomycin, etc.), elastin, repeat units thereof, structural units thereof, or combinations thereof. In several embodiments, one or more polymers are crosslinked to form the scaffold. In several embodiments, the polymers are not crosslinked to form the scaffold. In several embodiments, the scaffolds allow the efficient reduction in viability and/or eradication of microbes (e.g., prokaryotic cells, bacteria, protozoa, fungi, algae, amoebas, slime molds, etc. and in particular such microbes that have developed at least some degree of drug resistance) with low toxicity native tissue and patient cells (e.g., eukaryotic cells, mammalian cells, human cells, etc.).

One aspect of the present invention is pharmaceutical compositions that taking advantage of the synergistic antimicrobial properties of NO and gallium. Synergy is a concept known in the art and methods for determining synergy are provided below. A minireview of antimicrobial synergy measurements was prepared by Christopher Dern of Virginia Commonwealth University and can be found at jcm.asm.org/content/jcm/52/12/4124.full.pdf which is incorporated herein by reference in its entirety.

In illustrative embodiments, disclosed is a pharmaceutical composition comprising a nitric oxide releasing compound and an aqueous solution comprising gallium. In one embodiment, the nitric oxide releasing compound is a diazeniumdiolate or a nitrosothiol. In one embodiment, the gallium is at least partially complexed in the aqueous solution with a citrate moiety. The aqueous solution may also comprise chloride ions. These chloride ions remain from dissolving, for example, gallium chloride in solution. The aqueous solution may also comprise nitrate ions. These nitrate ions remain from dissolving, for example, gallium nitrate in solution. Citrate or other chelating agents, as described herein, may be included in the formulation for stabilizing gallium against the formation of gallium hydroxide, which is not highly soluble in water and thus may precipitate. In an embodiment, a form of gallium is gallium nitrate.

Furthermore, antimicrobial compositions comprising gallium and a nitric oxide-releasing cyclodextrin and their use in decreasing microbial load are provided herein. The inventors of the present application have surprisingly discovered that not only are compositions comprising gallium and nitric oxide donors effective against plankton bacteria and biofilms, but these compositions also exhibit synergistic effects against the same.

Provided herein are compositions comprising gallium and NO donors and scaffolds, and methods of treating various pathophysiologies using such compositions that leverage the synergistic effects of gallium combined with NO donors and scaffolds, which in turn leverage enhanced NO-release characteristics and beneficial physical properties, harnessing the abundant potential of NO-releasing pharmacological compounds and compositions. In several embodiments, provided herein are compositions that are highly efficacious as antimicrobials. In several embodiments, provided herein are compositions with beneficial antimicrobial properties. In several embodiments, the polymers and/or scaffolds disclosed herein have advantageous activity as mucoadhesive agents or chelating agents as described herein.

The gallium may be gallium (III), gallium nitrate (Ga(NO₃)₃), gallium chloride, a pharmaceutically acceptable salt, pharmaceutically acceptable complex, or combination thereof.

The composition may comprise about 0.001 mg to 100 mg of gallium. For example, the composition may comprise about 0.001, 0.002, 0.003, 0.004, 0.005, 0.006, 0.007, 0.008, 0.009, or 0.01 mg of gallium. The composition may comprise about 0.01, 0.02, 0.03, 0.04, 0.05, 0.06, 0.07, 0.08, 0.09, or 0.1 mg of gallium. The composition may comprise about 0.1, 0.2, 0.3, 0.4, 0.5, 0.6, 0.7, 0.8, 0.9, or 1.0 mg of gallium. The composition may comprise about 1, 2, 3, 4, 5, 6, 7, 8, 9, or 10 mg of gallium. The composition may comprise about 10, 20, 30, 40, 50, 60, 70, 80, 90, or 100 mg of gallium.

The composition may comprise gallium dosed at about 0.001 mg to 100 mg of gallium per kg of the patient. The composition may comprise gallium dosed at about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 mg of gallium per kg of the patient.

The gallium may be in the form of a pharmaceutically acceptable salt or pharmaceutically acceptable complex. The pharmaceutically acceptable salt may be nitrate, citrate, chloride, acetate, isocitrate, tartrate, or a mixture thereof. The pharmaceutically acceptable complex agent, which is also described herein as a chelating agent, may be mannitol, maltolate or a derivative, protoporphyrin IX or a derivative, lactoferrin, transferrin, ferritin, bacterial siderophores belonging to the catecholate, hydroxamate, and hydroxycarboxylate groups, bacterial hemophores, and any chelators of iron.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

It is contemplated that various combinations or sub-combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering an NO-donating composition” include “instructing the administration of an NO-donating composition.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 one millipascal-second” includes “10 one millipascal-second.”

The present invention is further defined in the following Examples. It should be understood that these Examples, while indicating preferred embodiments of the invention, are given by way of illustration only.

### EXAMPLES

### 2,6 cis-dimethylpiperdine-NONOate Synthesis

A stainless-steel vessel rated for high pressures is required for this reaction. 2,6-cis-dimethylpiperdine (DMP) is weighed into an appropriately sized container. Water is added to the container along with 2 to 4 equivalents of base (NaOH). Mix to dissolve the solids.

Transfer the solution into the high-pressure vessel and purge the headspace with nitrogen. Mix the solution and set nitric oxide pressure to 25 bar (temp 20° C.). Allow the reaction to run for minimum of 3 days. Stop the reaction by venting the NO. Precipitate the reaction product in acetone (10:1 acetone:reaction mixture). Filter the precipitate and vacuum dry. The material was found to have the following characteristics:
NO Total 3.7
(mol/mg):
T_{1/2}(h): 1.3
Max Flux (ppb/mg): 4933

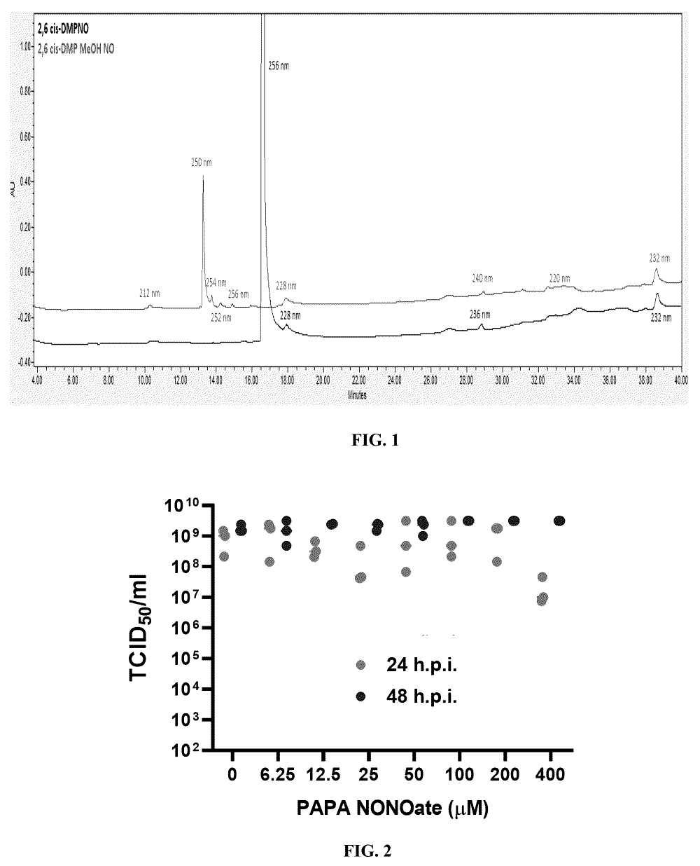

An HPLC of the product, run with two different eluents, shows a single peak for the product (FIG. 1).

### Antiviral Activity

The goal was to evaluate our therapeutics as an antiviral against SARS-CoV-2. The antiviral impact of PAPA NONOate and SNAP against SARS-CoV-2 was tested by exposing SARS-CoV-2 to a 2-fold dilution series of PAPA NONOate and SNAP in a series of separate experiments.

We evaluated the antiviral effect of PAPA NONOate with a single treatment at specified doses at 1-hour post-infection with MOI 0.01, including a titer of the SARS-CoV-2 stock virus and the dilution for MOI 0.01 at the start of the experiment. The results are shown in FIG. 2.
1. Vero E6 cells were infected with SARS-CoV-2 at the indicated MOI of 0.01.
2. At 1 hr post-infection virus was removed and exposed to drug compositions as follows:

PAPA NONOate was prepared ice cold 1× PBS to achieve 4 mM, which is a 10× stock. Further dilutions were made to 4 mM, 2 mM, 1 mM, 0.5 mM, 0.25 mM, 0.125 mM, 0.0625 mM. To achieve final concentrations for use. Add 100 μl of appropriate 10× stock to 900 μl of room temperature media. This should be done in one volume for all wells receiving that dose (e.g. for the 400 μM final dose with 3 replicates prepare 4 times the amount 400 μl 4 mM+3600 μl media). After each dose preparation in media add drug immediately to cells receiving that dose (e.g. add 1 ml of the 400 μM solution to a single well).

The final dose curve for PAPA NONOate was 400 μM, 200 μM, 100 μM, 50 μM, 25 μM, 12.5 μM, 6.25 μM, and 0 μM. All samples will be compared to the 0 μM dose, which is media alone.

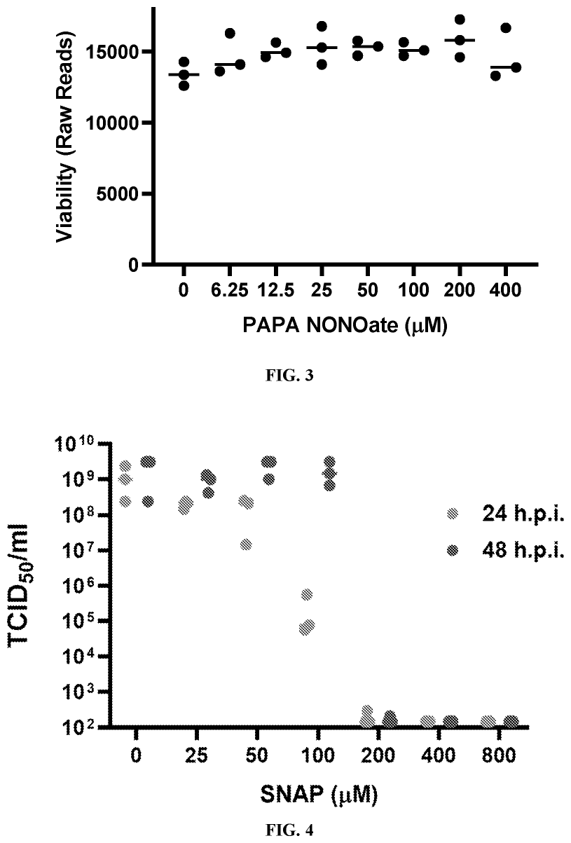

Referring now to the FIG. 2, the results from a standard TCID₅₀ titration, or plaque assays, of virus harvested at 24-hours and 48-hours post-infection is provided for PAPA NONOate at the specified concentrations. Referring now to FIG. 3, shown is a plot of the viability (raw reads) vs PAPA NONOate concentration (micromolar).

SNAP was prepared ice cold 1× PBS to prepare a 10× stock at 8 mM. Further dilutions were made to 8 mM, 4 mM, 2 mM, 1 mM, 0.5 mM, and 0.25 mM. To achieve final concentrations for use. Add 1001 of appropriate 10× stock to 900 μl of room temperature media. This should be done in one volume for all wells receiving that dose (e.g. for the 500 μM final dose with 3 replicates prepare 4 times the amount 400 μl 5 mM+3600 μl media). After each dose preparation in media add drug immediately to cells receiving that dose (e.g. add 1 ml of the 500 μM solution to a single well). The final dose curve for SNAP is 800 μM, 400 μM, 200 μM, 100 μM, 50 μM, 25 μM, and 0 μM.

All samples will be compared to the 0 μM dose, which is media alone. The surpernatant containing virus was harvested 24- and 48-hours post-infection. The results from a standard TCID₅₀ titration, or plaque assays, of virus harvested at 24-hours and 48-hours post-infection for SNAP at the specified concentrations is shown in FIG. 4.

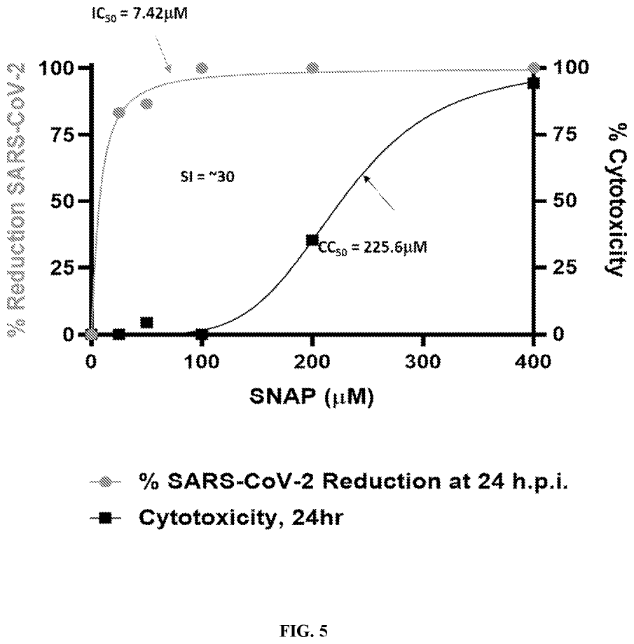

A plot of the % Reduction SARS-CoV-2 on a first y-axis and % Cytotoxicity on a second y-axis, demonstrating that SNAP has a SI of approximately 30 towards SARS-CoV-2, is shown in FIG. 5.

### Antimicrobial Activity

Having ascertained the MIC and MBC of many NO materials across many different pathogenic bacterial species, we sought to establish the synergies created according to compositions of the present disclosure.

### Method: Checkerboard Synergy Assay

Determined the MIC of each test article individually against the strains being tested using the CLSI MIC/MBC method.

Test articles are added to a 96-well plate so that when a 2× bacterial culture (prepared to 10⁶ CFU/ml) is added, the final concentration in all wells was 1×.

All test articles are prepared at 4×→diluted 1:1 when other TA is added (2×), and diluted 1:1 again to 1× when bacteria are added

Plates were incubated at 37° C. for 18-24 h.

### Data Analysis:

The lowest drug concentration in the array that did not support bacterial growth nor change color after incubation with Resazurin was determined the most effective inhibitory concentration.

The fractional inhibitory concentration index (FICI) was calculated using the equation below:

$$FICI = \frac{{MIC}_{AB}}{{MIC}_{A}} * \frac{{MIC}_{BA}}{{MIC}_{B}}$$

where MIC_{A} and MIC_{B} are the values determined for agents A and B in the MIC assays, respectively, and MIC_{AB} and MIC_{BA} are the concentrations of agent A and B that constituted the most effective inhibitory combination. Interpretations for the FICI value are shown below:

| FICI | Interpre- | |
|---|---|---|
| Value | tation | The Activity of the Combination is |
| <0.25 | Highly | Much greater than the sum of the activity of each |
| | synergistic | drug |
| ≤0.5 | Synergistic | Greater than the sum of the activity of each drug |
| ≤1 | Additive | Equal to the sum of the activity of each drug |
| ≤4 | Indifferent | The same as the activity of just one of the drugs |
| >4 | Antagonist | Lower than the activity of just one of the drugs |

### Method: Combination MIC/MBC Assay

### Purpose:

Combination MIC/MBC assays are useful when you want to look at only a few concentrations of one compound (Gallium citrate) combined with many concentrations of the second compound (BIOC76).

This method also allows us to see the change in both the MIC and MBC of a drug when in combination with another drug. Checkerboard assays only allow us to see changes in the MIC's.

### Method:

Test articles are added to a 96-well plate so that when a 2× bacterial culture (prepared to 10⁶ CFU/ml) is added, the final concentration in all wells was 1×.

All test articles are prepared at 4×→diluted 1:1 when other TA is added (2×) and diluted 1:1 again to 1× when bacteria are added.

TA #1 (Gallium citrate) is prepared and added to entire rows of a plate at a single concentration.

TA #2 (BIOC76) in then prepared and serially diluted in TA #1, so there are 2-fold dilutions of TA #2 combined with a single concentration of TA #1.

Plates were incubated at 37° C. for 18-24 h.

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | MC | 64.00 | 32.00 | 16.00 | 8.00 | 4.00 | 2.00 | 1.00 | 0.50 | 0.25 | 0.13 | GC |
| C | MC | 64.00 | 32.00 | 16.00 | 8.00 | 4.00 | 2.00 | 1.00 | 0.50 | 0.25 | 0.13 | GC |
| D | MC | 32.00 | 16.00 | 8.00 | 4.00 | 2.00 | 1.00 | 0.50 | 0.25 | 0.13 | 0.06 | GC |
| E | MC | 32.00 | 16.00 | 8.00 | 4.00 | 2.00 | 1.00 | 0.50 | 0.25 | 0.13 | 0.06 | GC |
| F | MC | 32.00 | 16.00 | 8.00 | 4.00 | 2.00 | 1.00 | 0.50 | 0.25 | 0.13 | 0.06 | GC |
| G | MC | 32.00 | 16.00 | 8.00 | 4.00 | 2.00 | 1.00 | 0.50 | 0.25 | 0.13 | 0.06 | GC |
| H | | | | | | | | | | | | |

B/C—BIOC76 alone (VT-20-063)
D/E—BIOC76 + 1 mg/ml Gallium citrate
F/G—BIOC76 + 0.5 mg/ml Gallium citrate
Concentrations show mg/ml of BIOC76
MC = media control
GC = growth control

### Data Analysis:

The ΔMIC/IBC is used to show the change in the MIC/IBC of BIOC76 when in combination with Gallium citrate (compared to when BIOC76 is used alone).

ΔMIC/MBC is the fold-decrease in BIOC76 concentration as a result of the combination.

Synergy of NO Donors and Gallium Nitrate with P. aeruginosa

We sought to determine whether an NO donor and gallium nitrate have antimicrobial synergy against a lab strain of P. aeruginosa (PAK). The Checkerboard assay (described herein) was used with a hepta-substituted cyclodextrin-based NO donor (hereinafter referred to as BIOC76) having approximately ˜4.7 μmol NO/mg dry powder.

Experimental Design: The MIC of BIOC76 and Gallium nitrate individually, then set up a checkerboard assay testing 49 combinations of BIOC76 and Gallium nitrate concentrations against 10⁶ CFU/ml P. aeruginosa strain PAK. Plates were incubated at 37° C. for 18-24 h, and growth was assessed visually. The following results were observed:

| | | MIC_{BIOC76} | MIC_{Ga(NO3)3} | MIC_{BIOC76} | MIC_{Ga(NO3)3} | | |
|---|---|---|---|---|---|---|---|
| | | alone | alone | combo | combo | | |
| Strain | Rep | (mg/ml) | (mg/ml) | (mg/ml) | (mg/ml) | FICI | Interpretation |
| PAK | Rep 1 | 1 | 0.065 | 0.125 | 0.015 | 0.375 | Synergistic |
| | Rep 2 | 1 | 0.065 | 0.125 | 0.015 | 0.375 | Synergistic |

Synergy of an NO Donor and Gallium Nitrate with P. aeruginosa Biofilm

In this study, we established whether an NO Donor (BIOC76) and Gallium nitrate are effective against P. aeruginosa biofilms grown under aerobic conditions. We concluded that BIOC76 is effective against P. aeruginosa biofilms, but Gallium nitrate is not.

Experimental Design: P. aeruginosa was grown as biofilms on a peg lid at 37° C. for 18-24 h. Added BIOC76, Gallium nitrate, or Tobramycin (positive control) to biofilms and incubated for another 18-24 h. Disrupted biofilms and plated to determine remaining biofilm-associated CFU's. MBEC=concentration that gives 3-log reduction in biofilm. The following results were obtained:

| | | MBEC of | MBEC of | MBEC of |
|---|---|---|---|---|
| | | BIOC76 | Gallium nitrate | Tobra |
| Strain | Rep | (mg/ml) | (mg/ml) | (μg/ml) |
| PAK (N0049) | Rep 1 | 4 | >16 | 8 |
| | Rep 2 | 4 | >16 | 4 |

Synergy of an NO Donor and Gallium Citrate with P. aeruginosa

In this study, we established whether BIOC76 and gallium citrate have antimicrobial synergy against multiple strains of P. aeruginosa. We concluded that BIOC76 and gallium citrate act synergistically against lab and clinical isolate strains of P. aeruginosa. We determined the MIC of BIOC76 and gallium nitrate individually, then set up a checkerboard assay testing 49 combinations of BIOC76 and Gallium nitrate concentrations against 10⁶ CFU/ml P. aeruginosa. Plates were incubated at 37° C. for 18-24 h, and growth was assessed visually.

The following results were obtained:

| | | FICI | Interpretation | FICI | Interpretation |
|---|---|---|---|---|---|
| Strain | Phenotype | (rep 1) | (rep 1) | (rep 2) | (rep 2) |
| N0049 | Lab | 0.365 | Synergistic | 0.365 | Synergistic |
| | strain | | | | |
| N0059 | XDR, | 0.223 | Synergistic | 0.223 | Synergistic |
| | TobraR, | | | | |
| | CarbR | | | | |
| N0054 | Mucoid | 0.510 | Additive | 0.510 | Additive |

MIC/MBC of a NO donor and gallium citrate against many PA strains

In this study, we established the MIC/MiBC's of BIOC76 and gallium citrate alone against each of 21 P. aeruginosa strains. We concluded that BIOC76 and Gallium citrate alone are largely bacteriostatic and Gallium citrate does not kill any strain of P. aeruginosa. The activity of both compounds is also strain-dependent. The results are as follows (the species for each strain is Pseudomonas aeruginosa:

| | | BIOC76 | | Gallium citrate | |
|---|---|---|---|---|---|
| | | (mg/ml) | | (mg/ml) | |
| Strain | | MIC | MBC | MIC | MBC |
| Designation | Strain | Rep 1 | Rep 1 | Rep 1 | Rep 1 |
| N0047 | AR-BANK #103 | 2 | 8 | 0.25 | >2 |
| N0048 | ATCC 19143 | 0.5 | 2 | 2 | >2 |
| N0049 | PAK | 2 | 4 | 0.065 | >2 |
| N0050 | AR-BANK #0095 | 2 | 8 | 0.25 | >2 |
| N0051 | AU32077 | 1 | 4 | 0.25 | >2 |
| N0052 | AU25834 | 1 | 2 | >2 | NP |
| N0053 | AU30524 | 4 | 16 | 0.25 | >2 |
| N0054 | AU26773 | 2 | 16 | 0.125 | >2 |
| N0055 | AU22725 | 1 | >8 | 0.0625 | >2 |
| N0056 | AU22505 | 8 | 16 | 1 | >2 |
| N0057 | AU35400 | 8 | 32 | 0.065 | >2 |
| N0058 | AU32543 | 2 | 8 | 0.5 | >2 |
| N0059 | AR-BANK #0054 | 2 | >16 | 0.0325 | >2 |
| N0069 | AR-BANK #0090 | 2 | >16 | 0.125 | >2 |
| N0070 | AR-BANK #0092 | 2 | >16 | 0.0625 | >2 |
| N0071 | AR-BANK #0100 | 2 | >16 | 0.125 | >2 |
| N0072 | AR-BANK #0105 | 2 | >16 | 0.5 | >2 |
| N0079 | AR-BANK #0108 | 2 | >16 | 0.125 | >2 |
| N0080 | AR-BANK #0110 | 2 | >16 | 0.125 | >2 |
| N0081 | AR-BANK #0111 | 2 | >16 | 0.25 | >2 |
| N0099 | ATCC 29260 | 2 | 8 | 0.125 | >2 |
| N00100 | PAK mucA22 | | | | |

### Combination MIC/MBC of an NO Donor and Gallium Citrate—Dose Ratio/Dose Minimization

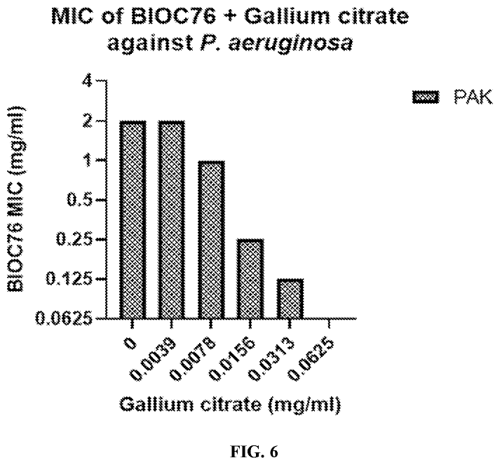

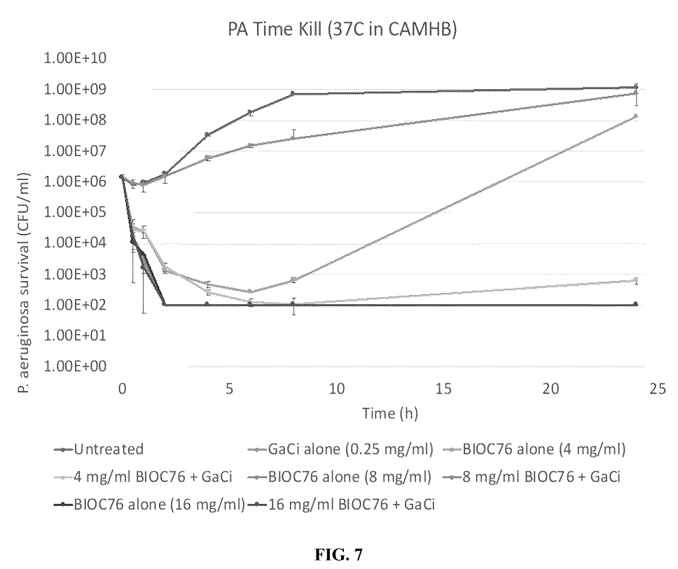

In this study, we established the minimal concentration of gallium citrate still exhibiting antimicrobial synergy with BIOC76 for P. aeruginosa. We concluded BIOC76 and gallium citrate maintain synergistic activity so long as the gallium concentration is at least about 0.005 to about 0.02 mg/ml. In this experimental, we set up MIC/MBC assay and serially diluted BIOC76 in rows with fixed concentrations of gallium citrate. Added P. aeruginosa (PAK) prepared at 10⁶ CFU/ml to wells and incubated at 37° C. for 18-24 h. ΔMIC/MBC=fold-decrease in BIOC76 concentration when combined with gallium citrate. The results are shown in FIG. 6, and in the following tables:

| | | MIC_{BIOC76} | MBC_{BIOC76} | Combo | Combo | | |
|---|---|---|---|---|---|---|---|
| | mg/ml | alone | alone | MIC_{BIOC76} | MBC_{BIOC76} | Δ | Δ |
| Strain | GaCi | (mg/ml) | (mg/ml) | (mg/ml) | (mg/ml) | MIC | MBC |
| N0049 | 2 | 2 | 4 | <0.06 | 2 | >2¹⁵ | 2 |
| | 1 | 2 | 4 | <0.06 | 4* | >2¹⁵ | 0* |
| | 0.5 | 2 | 4 | <0.06 | 2 | >2¹⁵ | 2 |
| | 0.25 | 2 | 4 | <0.06 | 2 | >2¹⁵ | 2 |
| | 0.125 | 2 | 4 | <0.06 | 4 | >2¹⁵ | 0 |
| | 0.065 | 2 | 4 | <0.06 | 4 | >2¹⁵ | 0 |
| | 0.0325 | 2 | 4 | 125 | 4 | 16 | 0 |
| | 0.0156 | 2 | 4 | 500 | 4 | 4 | 0 |

| | | | Combo | | |
|---|---|---|---|---|---|
| MIC_{BIOC76} | MIC_{GaCi} | GaCi | MIC_{BIOC76} | | |
| (mg/ml) | (mg/ml) | (mg/ml) | (mg/ml) | FICI | Effect |
| 2 | 0.065 | 0.0313 | 0.125 | 0.5625 | Additive |
| | | 0.0156 | 0.25-0.50 | 0.375-0.50 | Syner- |
| | | | | | gistic |
| | | 0.0078 | 1 | 0.62 | Additive |
| | | 0.0039 | 2 | 1.06 | Indifferent |

NO Donor and Gallium Citrate—Time-Kill Assay with P. aeruginosa (CAMB at 37° C.)

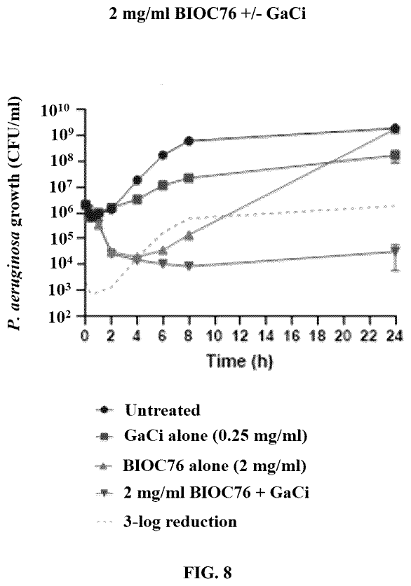

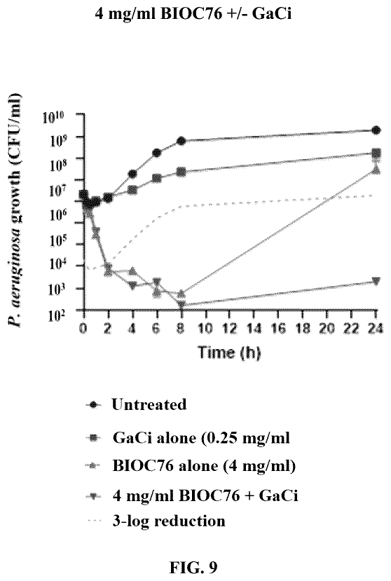

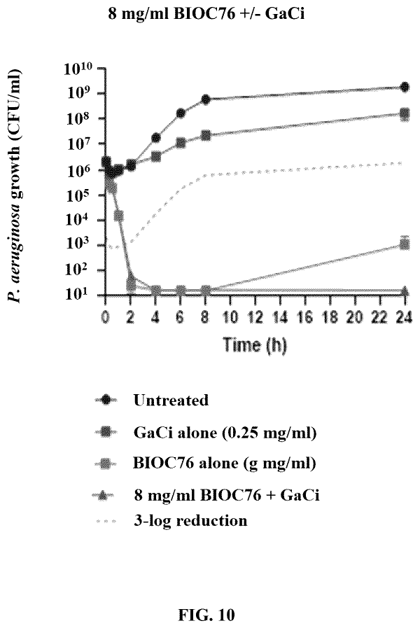

In this study, we established the viability of P. aeruginosa in a broth at 37° C. when exposed to BIOC76 alone compared to BIOC76+gallium citrate. We concluded that P. aeruginosa can recover after 24 h in broth when exposed to 4 mg/ml BIOC76 alone but adding 0.25 mg/ml Gallium citrate to the BIOC76 prevents this recovery, indicating there may be bactericidal synergy. Furthermore, gallium citrate enhances the bactericidal activity of the NO donor against P. aeruginosa at all concentrations tested, supporting the use of NO donor and gallium combination therapeutics. To conduct this experiment, we suspended P. aeruginosa (PAK) in CAMHB to a final concentration of 10⁶ CFU/ml. Bacteria were exposed to BIOC76, GaCi, or BIOC76+GaCi and incubated at 37° C. Bacterial survival was measured over 24 h and is shown in FIGS. 7-10.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above.

Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A pharmaceutical composition comprising a nitric oxide releasing compound and an aqueous solution, wherein the nitric oxide releasing compound is soluble to at least about 25 mg/ml in the aqueous solution at a physiologically compatible pH; the composition further comprising one or more of a chelating agent, a mucoadhesive agent, or a low molecular weight polyethylene glycol, wherein the nitric oxide releasing compound is a diazeniumdiolate or a nitrosothiol.

2. (canceled)

3. The pharmaceutical composition of claim 1, wherein the nitric oxide releasing compound has a NO release half-life at normal physiological temperature and pH of at least 15 minutes.

4. The pharmaceutical composition of claim 1, wherein the nitric oxide releasing compound is S-Nitroso-N-Acetyl-D,L-Penicillamine (SNAP).

5. The pharmaceutical composition of any of claim 1, wherein the nitric oxide releasing compound has a total releasable NO storage in a range of 1-10 μmol of NO per mg of compound, a NO half-life in the range of about 0.1-24 hours, a total duration of NO release in the range of 1-60 hours, or a total NO release after 4 hours in the range between 0.1-1.0 μmol of NO per mg of compound.

6-8. (canceled)

9. The pharmaceutical composition of any of claim 1, further comprising a pharmaceutically-acceptable carrier or excipient.

10. The pharmaceutical composition of any of claim 1, further comprising a siderophore.

11. The pharmaceutical composition of any of claim 1, wherein the pharmaceutical composition is formulated as a nebulizable formulation.

12. The pharmaceutical composition of any of claim 1, in the form of an inhalable powder, packaged in an inhaler.

13. A method of delivering nitric oxide to a subject in need of treatment comprising administering an effective amount of the pharmaceutical composition of claim 1 to the subject.

14. A method of treating a disease state comprising administering an effective amount of the pharmaceutical composition of claim 1 to a subject in need thereof.

15. The method of claim 14, wherein the disease state is a microbial infection.

16. The method of claim 14, wherein the disease state is a respiratory infection.

17. The method of claim 14, wherein the disease state is a viral respiratory infection.

18. The method of claim 14, wherein the viral respiratory infection is SARS-CoV-2.

19. The method of claim 14, wherein the diseases state involves the formation of a bacterial biofilm.

20. The method of claim 14, wherein the disease state is bacterial vaginosis, urinary tract infections, catheter infections, middle-ear infections, dental plaque, gingivitis, coating on contact lenses, cystic fibrosis, bronchiectasis, chronic obstructive pulmonary diseases (COPD), the patients on ventilators, or a combination thereof.

21. The method of claim 14, wherein the disease state is a cancer, a cardiovascular disease, a microbial infection, platelet aggregation and platelet adhesion caused by the exposure of blood to a medical device, pathological conditions resulting from abnormal cell proliferation, transplantation rejections, autoimmune diseases, inflammation, vascular diseases, scar tissue, wound contraction, restenosis, pain, fever, gastrointestinal disorders, respiratory disorders, sexual dysfunctions, sexually transmitted diseases, or a combination thereof.

22. A method of reducing or preventing microbial load on a surface comprising applying the pharmaceutical composition of claim 1 to a surface contaminated with a plurality of microbes; wherein the composition releases nitric oxide and induces oxidative and/or nitrosative damage to microbial DNA and membrane structures, thereby preventing or reducing microbial load, and wherein said plurality of microbes comprises two or more of the following: gram-positive bacteria, gram-negative bacteria, fungi, yeast, and viruses.

23. The method of claim 22, wherein the surface is an organic surface.

24. The method of claim 22, wherein the surface is a wound surface.

25. The method of claim 24, wherein the surface is animal skin, optionally human tissues.

26. (canceled)

27. The method of claim 22, wherein the surface is an inorganic surface.

28. The method of claim 27, wherein the inorganic surface is an external or internal surface of a medical device.

29. The method of claim 28, wherein application of the compound generates an anti-microbial coating on the external or internal surface of the medical device.

30. The method of claim 28, wherein the medical device comprises an endoscope, catheter, prosthetic joint, mechanical heart valve, pacemaker, a contact lens, endotracheal tube, intrauterine device, or a combination thereof.

31. (canceled)

32. The method of any claim 22, wherein the microbial load comprises drug-resistant bacteria.

33. The method of claim 32, wherein the microbial load comprises microbes associated with the presence of SARS-CoV-2, human immunodeficiency virus, herpes simplex virus, papilloma virus, parainfluenza virus, influenza, hepatitis, Coxsackie Virus, herpes zoster, measles, mumps, rubella, rabies, pneumonia, hemorrhagic viral fevers, H1N1, prions, parasites, fungi, mold, Candida albicans, Aspergillus niger, Escherichia coli, Pseudomonas aeruginosa, and Staphylococcus aureus, Group A streptococci, S. pneumoniae, Mycobacterium tuberculosis, Campylobacter jejuni, Salmonella, Shigella, carbapenem-resistant Enterobacteriaceae Methicillin-resistant Staphylococcus aureus, Burkholderia cepacia, and combinations thereof.

34. The method of claim 32, wherein the microbial load comprises microbes associated with the formation of a biofilm.

35. The method of claim 32, wherein the microbial load comprises Methicillin-resistant Staphylococcus aureus, carbapenem-resistant Enterobacteriaceae, Staphylococcus aureus, Pseudomonas aeruginosa, or Burkholderia cepacia.

36. A method for treating a patient suffering from cystic fibrosis, comprising administering an effective amount of a compound of claim 1 to the subject.

37. A method treating a patient with a biofilm comprising administering an effective amount of a compound of claim 1 to the subject, wherein the composition reduces the biofilm.

38. A method for inhibiting infection to host cells comprising administering the composition of claim 13 in an amount sufficient to establish binding to ACE2 receptors of said cells.

39. The composition of claim 13, wherein the siderophore has binding affinity to an ACE2 receptor.

40. A pharmaceutical product comprising a first component comprising an aqueous solution and a second component comprising a nitric oxide donor, wherein mixing the first component and the second components according to a predetermined ratio results in a therapeutic formulation having a pH between about 7 and 8, the therapeutic formulation further comprising one or more of a chelating agent, a mucoadhesive agent, or a low molecular weight polyethylene glycol.

41. A method of treating respiratory infection, the method comprising administering a pharmaceutical composition comprising a nitric oxide donor and a mucoadhesive agent so as to contact pathogens within the respiratory tract.

42. The method of claim 41, wherein the pharmaceutical composition is selected from an intranasal spray, an oral rinse, an otic solutions, or a nebulized solution.

43-75. (canceled)
